(19) United States
(12) Patent Application Publication
(10) Pub. No.: US 2010/0285002 A1
(43) Pub. Date: Nov. 11, 2010

(54) TREATMENT OR PREVENTION OF INFLAMMATION BY TARGETING CYCLIN D1

(75) Inventors: Dan Peer, Kiryat Ono (IL); Motomu Shimaoka, Brookline, MA (US)

Correspondence Address: DAVID S. RESNICK, NIXON PEABODY LLP, 100 SUMMER STREET, BOSTON, MA, 02110-2131, US

(73) Assignee: IMMUNE DISEASE INSTITUTE, INC., Boston, MA (US)

(21) Appl. No.: 12/811,229

(22) PCT Filed: Dec. 30, 2008
(86) PCT No.: PCT/US08/88523

§ 371 Date: Jun. 30, 2010

Related U.S. Application Data
(60) Provisional application No. 61/018,919, filed on Jan. 4, 2008.

(51) Int. Cl. A61K 39/395; A61K 31/7088; A61P 37/00; A61P 29/00; A61P 25/28; A61P 19/02

(52) U.S. Cl. 424/130.1; 514/44 R; 514/44 A

(57) ABSTRACT

In one aspect, the invention relates to the treatment and/or prevention of inflammation by inhibition of cyclin D1. In one embodiment, Th1-mediated inflammation is selectively inhibited or reduced by a method comprising administering an agent that inhibits cyclin D1. In another embodiment, an autoimmune disease or a disorder characterized by or involving a Th1 inflammatory response is treated or prevented in a subject by a method comprising administering to the subject an agent that inhibits cyclin D1.

### CROSS REFERENCE TO RELATED APPLICATION

This application claims benefit under 35 U.S.C. §119(e) of the U.S. Provisional Application No. 61/018,919 filed Jan. 4, 2008, the contents of which are incorporated herein by reference in its entirety.

### GOVERNMENT SUPPORT

This invention was made with Government support under Grant No.: AI63421 awarded by the National Institutes of Health. The Government has certain rights in the invention.

### BACKGROUND OF INVENTION

Cyclin D1 is an important cell cycle regulating molecule and an established target for cancer therapy (Lee & Sicinski, 2006, Cell Cycle 5: 2110-2114; Stacey, 2003, Curr. Opin. Cell Biol. 15: 158-163). By binding to cyclin-dependent kinases (CDKs), cyclin D1 serves as a key sensor and integrator of extracellular signals of cells in early to mid G1 phase to drive cell proliferation. As cancers are characterized with uncontrolled and infinite cell proliferation, the aberrant regulation of cyclin D1 is implicated in malignant cell transformation and growth of many cancer cells. Therefore, cyclin D1 has been seen as a promising therapeutic target for cancer therapies.

A canonical (CDK-dependent) cyclin D1 pathway is mediated by its binding to CDK 4 and 6, leading to phosphorylation of retinoblastoma protein (Rb) that liberates E2F transcription factor and, thereby, lets the cell cycle proceed. More recently, non-canonical cyclin D1 pathways have been identified in which cyclin D1 functions in a CDK-independent manner. For example, cyclin D1 directly interacts with several transcriptional activators and repressors, playing important roles in the regulation of gene expression, metabolism, and cell migration. Rossi et al. (2006, Nature Med. 12: 1056-1064) and Zoja et al. (2007, Arthritis Rheum. 56: 1629-1637) report the targeting of CDKs for anti-inflammation.

Cyclin D1 was previously thought not to be expressed in normal lymphocytes. However, recent investigations have revealed that normal lymphocytes do express cyclin D1. van Dekken et al. (2007, Acta Histochemica 109: 266-272) reported upregulation of cyclin D1 and downregulation of the tumor suppressor E-cadherin occurs in the pre-malignant state in ulcerative colitis (UC). The authors concluded that this may contribute to the high potential for malignant degeneration of dysplasia in UC-related colitis. Yang et al. (2006, Cell Cycle 5: 180-183) examined contributions of D-type cyclins to proliferation in the mouse intestine. The authors reported that Cyclin D1 mRNA increased in the dextran sulfate sodium (DSS)-induced colitis model. Yang et al. reported that, in addition to epithelial cells, inflammatory cells in the mesenchyme and lymphoid aggregates were positive for cyclin D1 protein. The authors concluded that the D type cyclins are differentially regulated after stress in the intestine. Wong et al. (2003, Hum. Pathol. 34: 580-588) report examination of changes in cyclin D1 and p21^{Waf1/CIP1} expression along the UC-related dysplasia-carcinoma sequence. The authors reported increased expression of cyclin D1 in active UC compared with quiescent UC.

### SUMMARY OF THE INVENTION

The invention relates to the discovery that cyclin D1 blockade leads to the suppression of two distinct pathways critical for the pathogenesis and/or progression of inflammation. In one pathway, cyclin D1 blockade results in suppression of aberrant cellular proliferation of mononuclear leukocytes in inflammation in a CDK-dependent manner. In the other pathway, cyclin D1 blockade selectively suppresses pro-inflammatory Th1 cytokines (e.g., TNF-α and IL-12), but not anti-inflammatory Th2 cytokines (e.g., IL-10) in a CDK-independent manner. Inhibition of cyclin D1, but not inhibition of CDKs can interfere with the cell-cycle independent (i.e., the CDK-independent) pathway, leading to the suppression of pro-inflammatory Th1 cytokines. Cyclin D1 is thus identified as a target for the treatment or prevention of inflammation. Cyclin D1 blockade is shown herein to inhibit the pathology of ulcerative colitis in an in vivo model of the disease. Cyclin D1 is thus identified as a target for the treatment or prevention of inflammatory bowel disease, and other autoimmune diseases, particularly those in which Th1 pro-inflammatory cytokines mediate the inflammatory pathology.

In one aspect, described herein is the use of an agent that inhibits cyclin D1 for the preparation of a medicament for the treatment or prevention of inflammation in a subject in need thereof, wherein administering said agent reduces or prevents inflammation in a said subject.

In another aspect, described herein is the use of an agent that inhibits cyclin D1 for the preparation of a medicament for the treatment or prevention of Th1-mediated inflammation in a subject in need thereof, wherein administering said agent reduces or prevents Th1-mediated inflammation in a said subject.

In another aspect, described herein is the use of an agent that inhibits cyclin D1 for the preparation of a medicament for the treatment of an autoimmune disease or a disorder characterized by or involving a Th1 inflammatory response in a subject in need thereof, wherein administering said agent to said subject reduces said Th1 inflammatory response.

In another aspect, described herein is the use of an agent that inhibits cyclin D1 for the treatment or prevention of inflammation in a subject in need thereof, wherein administering said agent reduces or prevents inflammation in a said subject.

In another aspect, described herein is the use of an agent that inhibits cyclin D1 for the treatment or prevention of Th1-mediated inflammation in a subject in need thereof, wherein administering said agent reduces or prevents Th1-mediated inflammation in a said subject.

In another aspect, described herein is the use of an agent that inhibits cyclin D1 for the treatment of an autoimmune disease or a disorder characterized by or involving a Th1 inflammatory response in a subject in need thereof, wherein administering said agent to said subject reduces said Th1 inflammatory response.

In one aspect, described herein is a method of treating or preventing inflammation, the method comprising administering an agent that inhibits cyclin D1 to an individual in need thereof.

In another aspect, described herein is a method of selectively inhibiting Th1-mediated inflammation, the method comprising administering an agent that inhibits cyclin D1 to a subject in need thereof, wherein Th1-mediated inflammation is inhibited. The method can comprise a step of testing an individual in need of treatment for the level or expression of a Th1 cytokine; an elevated level of at least one such Th1 cytokine indicates that the subject would benefit therapeutically or prophylactically from cyclin D1 blockade or inhibition.

In one embodiment of these and all other aspects described herein, the administration of an agent reduces the expression of Th1 cytokines. Th1 cytokines include, but are not limited to TNF-α, IL-2, IL-12, IFN-γ, and IL-23.

In another embodiment of these and all other aspects described herein, the agent comprises an antibody, a nucleic acid or a small molecule. Where the agent comprises a nucleic acid, the nucleic acid can be, for example, an interfering RNA, e.g., an siRNA or RNAi molecule or other double-stranded RNA-based nucleic acid inhibitor of gene expression, e.g., an miRNA, etc. The double-stranded RNA-based nucleic acid inhibitors mediate the degradation of mRNA encoding the target gene, in this instance, cyclin D1 mRNA.

In another embodiment of these and all other aspects described herein, the agent comprises a targeting moiety. Targeting moieties can, for example, target an agent to a particular cell type, e.g., a leukocyte, including, but not limited to a lymphocyte, monocyte, macrophage or any other desired cell type. Where a leukocyte is targeted, the targeting moiety can, for example, bind to a cell-surface molecule, e.g., an integrin molecule expressed on the target cell, e.g., B7 expressed on a target lymphocyte.

In another aspect, described herein is a method of treating an autoimmune disease or inflammatory disorder characterized by or involving Th1-mediated inflammation, in a subject in need thereof. The method comprises administering to the subject an agent that inhibits Cyclin D1, wherein the Th1-mediated inflammation is reduced. In one embodiment, the subject is tested for the expression or presence of a Th1 cytokine; an elevated level of one or more such Th1 cytokines indicates that the subject would benefit therapeutically or prophylactically from cyclin D1 blockade or inhibition. Cyclin D1 inhibition preferably reduces the level of at least one Th1-mediated cytokine or its expression. In another embodiment, the at least one Th1 cytokine includes but is not limited to one or more of TNF-α, IL-2, IL-12, IFN-γ, and IL-23.

In one embodiment, the autoimmune disease or inflammatory disorder includes, but is not limited to an inflammatory bowel disease, ulcerative colitis, Crohn's disease, celiac disease, autoimmune hepatitis, chronic rheumatoid arthritis, psoriatic arthritis, insulin-dependent diabetes mellitus, multiple sclerosis, Alzheimer's disease, enterogenic spondyloarthropathies, autoimmune myocarditis, psoriasis, scleroderma, myasthenia gravis, multiple myositis/dermatomyositis, Hashimoto's disease, autoimmune hypocytosis, pure red cell aplasia, aplastic anemia, Sjogren's syndrome, vasculitis syndrome, systemic lupus erythematosus, glomerulonephritis, pulmonary inflammation (e.g., interstitial pneumonia), septic shock and transplant rejection.

In another embodiment, the agent comprises an antibody, a nucleic acid or a small molecule. Where the agent comprises a nucleic acid, the nucleic acid can be, for example, an interfering RNA, e.g., an siRNA or RNAi molecule or other double-stranded RNA-based nucleic acid inhibitor of gene expression, e.g., an miRNA, etc. The double-stranded RNA-based nucleic acid inhibitors mediate the degradation of mRNA encoding the target gene, in this instance, cyclin D1 mRNA.

In another embodiment, the agent comprises a targeting moiety. Targeting moieties can, for example, target an agent to a particular cell type, e.g., a lymphocyte or any other desired cell type. Where a lymphocyte is targeted, the targeting moiety can, for example, bind an integrin molecule expressed on the target lymphocyte, e.g., B7.

As used herein, the term “selectively,” when applied to the inhibition of a Th1-mediated immune response or inflammation means that the production or level of Th2 cytokines is not inhibited.

As used herein, the term “inhibits” or “inhibition” refers generally to at least a 10% reduction in an activity or amount. As an example, an agent that “inhibits” cyclin D1 reduces the level or an activity of cyclin D1 by at least 10%, and preferably by at least 20%, 30%, 40%, 50%, 60%, 70%, 80%, 90%, 95%, 97%, 99% or even by 100% (i.e., complete inhibition). Similarly, the term “reduces” refers to an at least 10% reduction in a given quantity or property relative to a reference, preferably at least a 20%, 30%, 40%, 50%, 60%, 70%, 80%, 90%, 95%, 97%, or 99% reduction, and most preferably a 100% reduction (i.e., complete reduction).

As used herein, the term “inhibits cyclin D1” means that the expression or Th1-activating activity of cyclin D1 is inhibited as that term is defined herein. An agent that inhibits cyclin D1 is preferably selective for cyclin D1 inhibition. That is, where an agent that kills a cell or arrests the cell cycle might be viewed as ultimately inhibiting cyclin D1 or all cell-cycle-related activities, such an agent, which acts proximally on another pathway or pathways, would not be viewed as a “cyclin D1 inhibitor.” To be clear, agents that inhibit the expression of cyclin D1 by, e.g., interfering with transcription or translation of cyclin D1 mRNA are encompassed by the term “cyclin D1 inhibitor.”

As used herein, the term “Th1 cytokine” refers to a cytokine produced by a T helper 1 or Th1 cell.

As used herein, the term “antibody” refers to an immunoglobulin molecule that binds a known target antigen. The term refers to molecules produced in vivo as well as those produced recombinantly, and refers to monoclonal and polyclonal antibodies. The term encompasses not only full-length, multi-subunit antibodies most often found in vivo, but also antigen-binding fragments and constructs derived from or based upon an antigen-binding immunoglobulin. Thus, the term “antibody” encompasses antigen-binding fragments such as a Fab, Fab′, F(Ab)′₂ and scFv fragments, as well as, for example, an antigen-binding variable domain, e.g., a V_{H} or V_{L} domain (often referred to as “single domain antibodies”). An “antibody” as the term is used herein can include a fusion of an antigen-binding polypeptide with a non-antibody polypeptide, as well as a dual- or bi-specific antibody construct or multivalent constructs. The technology for preparing and isolating antibodies that specifically bind a given target is well known to the ordinarily skilled artisan, as are techniques for preparing modified versions of or constructs containing antibodies as the term is used herein.

As used herein, the term “small molecule” refers to a chemical agent including, but not limited to peptides, peptidomimetics, amino acids, amino acid analogs, polynucleotides, polynucleotide analogs, aptamers, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

An “RNA interfering agent” as used herein, is defined as any agent which interferes with or inhibits expression of a target gene or genomic sequence by RNA interference (RNAi). Such RNA interfering agents include, but are not limited to, nucleic acid molecules including RNA molecules which are homologous to the target gene or genomic sequence, or a fragment thereof, short interfering RNA (siRNA), short hairpin or small hairpin RNA (shRNA), miRNAs and small molecules which interfere with or inhibit expression of a target gene by RNA interference (RNAi).

As used herein, the term “targeting moiety” refers to a moiety that specifically binds a marker expressed by a cell or tissue type one wishes to target with an agent, e.g., an inhibitory agent. In practice, a “targeting moiety” is distinct from, but physically associated with the agent one wishes to direct to a target. Targeting moieties can include, as non-limiting examples, receptors, ligands, aptamers, proteins or binding fragments thereof, and antibodies or antigen-binding fragments thereof.

As used herein, the term “specifically binds” refers to binding with a dissociation constant (k_{d}) of 100 μM or lower, e.g., 75 μM, 60 μM, 50 μM, 40 μM, 30 μM, 20 μM, 10 μM, 1 μM, 100 nM, 50 nM, 10 nM, 1 nM or less.

### BRIEF DESCRIPTION OF THE DRAWINGS

FIG. 1. The processes involved in generating I-tsNP. Multi-lamellar vesicle (MLV) prepared (as described in Methods) is extruded to form a uni-lamellar vesicle (ULV) with a diameter of ˜100 nm. Hyaluronan is covalently attached to DPPE in ULV. An antibody to the integrin is covalently attached to hyaluronan, generating I-tsNP. siRNAs are entrapped by re-hydrating lyophilized β7 I-tsNP with water containing protamine-condensed siRNAs.

FIG. 2. β7 I-tsNP delivers siRNAs to silence in leukocytes in a β7-specific manner. (A) Cy3-siRNA delivery via β7 I-tsNP to WT, but not to β7 knockout (KO), splenocytes as revealed by flow cytometry. (B) Confocal microscopy with DIC morphologies showing the β7 integrin-specific intracellular delivery of Cy3-siRNA. Images were acquired 4 h after addition to splenocytes of naked Cy3-siRNA, or Cy3-siRNA in Alexa 488-labeled β7 I-tsNP or IgG-sNP. (C) Ku70-siRNA delivery with β7 I-tsNP induced silencing. Splenocytes were treated for 48 h with 1,000 pmol Ku70- or control luciferase (Luci)-siRNAs, delivered as indicated. (D) In vivo silencing of Ku70 in mononuclear cells from the gut and spleen of WT, but not KO, mice. siRNAs (2.5 mg/Kg) entrapped as indicated were intravenously injected. Seventy-two h after injection, Ku70 expression was examined. Ku70 protein expression was determined by immunofluorescent cytometry following cell permeabilization and expressed as % of Ku70 expression in mock (C & D). Data are expressed as the mean±SEM of at least three independent experiments (A, C, D). p<0.05*, 0.01† vs. mock. (E) Bio-distribution of 3H-cholesterylhexadecylether (3H-CHE)-labeled nanoparticles in mice with or without DSS-induced colitis. Pharmacokinetics and bio-distribution were determined 12 h after injection in a total of 6 mice/group in three independent experiments. Blood half-lives of β7 I-tsNP in healthy and diseased mice were 4.3 and 1.8 h, respectively. p<0.01†

FIG. 3. Silencing of CyD1 by siRNA delivery with β7 I-tsNP and its effects on cytokine expression. (A). Silencing of CyD1 (measured by a real-time quantitative RT-PCR) and its effects on proliferation (measured by [3H]-thymidine incorporation). In in vitro treatments, splenocytes were examined after 72 h incubation with 1,000 pmol siRNAs delivered as indicated in the presence or absence of CD3/CD28 stimulation. In in vivo treatments, siRNAs (2.5 mg/Kg) entrapped as indicated were intravenously injected into a total of 6 mice per group in three independent experiments. Seventy-two h later, mononuclear cells harvested from the gut and spleen were examined. p<0.05*, 0.01† vs. mock-treated samples. (B) CyD1-knockdown selectively suppresses Th1 cytokine mRNA expression in splenocytes activated via CD3/CD28 (C) CyD1-knockdown selectively suppresses Th1 cytokine mRNA expression independently of its inhibitory effects on cell cycle. In aphidicolin-treated TK-1 cells in which cell cycle was arrested, PMA/iomomycin-upregulated Th1 cytokine mRNA expression was selectively suppressed by CyD1-knockdown. (D) Cell cycle-independent suppression of Th1 cytokines observed with individual applications of 4 different CyD1-siRNAs (C & D) TK-1 cells pretreated for 12 h with aphidicolin were treated with siRNAs (1,000 pmol) delivered as indicated for another 12 h in the presence of PMA/ionomycin and aphidicolin. (B-D) p<0.05*, 0.01† v.s. mock-treated activated cells (A-D) mRNA levels for CyD1 and cytokines were measured by a real-time quantitative RT-PCR. Data are expressed as the mean±SEM of at least three independent experiments.

FIG. 4. Cyclin-D1-siRNA delivered by β7 I-tsNP alleviated intestinal inflammation in DSS induced colitis. Mice were intravenously administered CyD1- or luciferase-siRNAs (2.5 mg/Kg) entrapped in either β7 I-tsNP or IgG sNP, or naked CyD1-siRNA (2.5 mg/Kg) at days 0, 2, 4, and 6 (a total of 6 mice/group in three independent experiments). (A) Changes in body weight. (B) Hematocrit (HCT) values measured at day 9. (C) Representative histology at day 9 (haematoxylin and eosin staining, 100×). (D) mRNA expression of CyD1 and cytokines in the gut. mRNA expression was measured by quantitative RT-PCR with homogenized colon samples harvested at day 9. Data are expressed as the mean values±SEM of three independent experiments (A, B, D). p<0.05*, p<0.01† v.s. mock-treated DSS-mice

FIG. 5. Covalent attachment of antibodies to hyaluronan-coated nanoparticles abolishes their binding to CD44. Unmodified hyaluronan binds the receptor CD44 (13). Of note, however, in the context of sNP, such activity disappeared when hyaluronan was covalently coupled to an antibody (i.e., FIB504). Thus, targeting specificity should depend only the given antibody involved. Binding of β7 I-tsNP and sNP entrapping fluorescein (5 μM) to CD44+ β7 integrin-B16F10 cells was examined using flow cytometry. Representative histograms of β7 I-tsNP (thick line), sNP (dashed line), and mock treatment (thin line) are shown.

FIG. 6. The presence of hyaluronan is critical to maintaining the ability of nanoparticles to bind β7 integrin during a cycle of lyophilization and rehydration. Nano-sized liposomes were surface-modified with either Alexa488-labeled antibodies alone (left panels) or hyaluronan (HA) and Alexa488-labeled antibodies (right panels). Binding to the β7 integrin on splenocytes was examined using flow cytometry before (dashed lines) and after (solid lines) samples had been subjected to a cycle of lyophilization and rehydration. Particles surface-modified with Alexa488-labeled control IgG serve as a negative control to show background binding (bottom panels). Note that after lyophilization/rehydraton, liposomes surface modified with hyaluronan and FIB504 mAb (top-right panel; i.e., β7 I-tsNP) retained the ability to bind splenocytes, whereas liposomes with FIB504 mAb alone lost the ability to bind (top-left panel).

FIG. 7. β7 I-tsNP delivers siRNAs to TK-1 cells. Confocal microscopy with DIC morphologies showing the intracellular delivery of Cy3-siRNA. Images were acquired 4 h after addition to TK-1 cells of naked Cy3-siRNA or Cy3-siRNA in Alexa 488-labeled β7 I-tsNP or IgG-sNP.

FIG. 8. Surface-modification with β7 integrin mAb as well as siRNA-entrapment are required to induce robust gene silencing in splenocytes. (A) To study whether or not hyaluronan, a ligand for CD44, was used to effectively silence in CD44high activated splenocytes, CyD1-siRNA entrapped in hyaluronan-nanoparticles that lack β7 integrin mAb (sNP entrapping CyD1-siRNA) was tested. sNP entrapping 1,000 pmol CyD1-siRNA induced little silencing in CD44high activated splenocytes, supporting the idea that β7 integrin mAb is necessary for intracellular siRNA delivery, a prerequisite to induce effective gene silencing. (A & B) To demonstrate that an entrapment, but not merely a surface-association, of siRNA to β7 I-tsNP is required for robust silencing, CyD1-siRNA that was surface-associated with β7 I-tsNP was studied. CyD1-siRNA that was surface-associated with β7 I-tsNP (β7 I-tsNP associated with CyD1-siRNA) was made by mixing a protamine-condensed CyD1-siRNA solution to fully water-rehydrated β7 I-tsNP; whereas CyD1-siRNA-entrapped in β7 I-tsNP (β7 I-tsNP encapsulating CyD1-siRNA) was made by rehydrating lyophilized β7 I-tsNP with a protamine-condensed CyD1-siRNA-containing solution. Note that β7 I-tsNP entrapping CyD1-siRNA exhibited ˜100 times as potent silencing as did β7 I-tsNP associated with CyD1-siRNA. (A & B) Splenocytes pre-treated for 12 h with PMA/iomomycin were incubated for another 12 h with CyD1-siRNA (A, 1,000 pmol; B, 10˜1,000 pmol) delivered as indicated. mRNA expression of was determined by a real-time quantitative RT-PCR, and normalized to that of GAPDH. Data are mean±SEM of at least three independent experiments. P<0.05*, 0.01† v.s. mock-treated activated cells.

FIG. 9. Entrapment in β7 I-tsNP protects siRNAs from inactivation by serum (A) and RNases (B). Because degradation of siRNAs by RNases present in serum has the potential to greatly undermine their activity during in vivo delivery (14, 15), the stability of siRNA entrapped in β7 I-tsNP to RNase exposure was examined. In contrast to naked Ku70-siRNA (1,000 pmol), which was inactivated following exposure to RNase A (20 ng/mL) or 50% serum, β7 I-tsNP-entrapped Ku70-siRNA (1,000 pmol) maintained its ability to silence a specific gene, demonstrating the protective properties of entrapment in β7 I-tsNP against RNase degradation. Ku70-siRNA entrapped in β7 I-tsNP was delivered to TK-1 cells as described in Methods. Naked Ku70-siRNA was delivered to TK-1 cells using an Amaxa™ nucleofection. Activities of Ku70-siRNA were studied by examining the efficacy of Ku70-knockdown 48 h after delivery. Note that β7 I-tsNP and Amaxa showed comparable Ku70 knockdown efficacies before exposure to FCS and RNase A. Data represent the percentage of Ku70 expressed by untreated cells, and are shown as the mean±SEM of three independent experiments.

FIG. 10. siRNA delivery with β7 I-tsNP does not induce the potential unwanted effects such as (A) cellular activation via the cross-linking of cell surface integrins by β7 I-tsNP and (B) the triggering of interferon responses, an issue common to siRNA applications (14, 15). (A) Expression of activation markers CD69 and CD25 on splenocytes measured by flow cytometry 48 h after treatment with 1 nmol luciferase-siRNA entrapped in β7 I-tsNP. FACS histogram overlays show cells treated with β7 I-tsNP entrapping siRNA (clashed lines), siRNA alone (thin lines), and PHA as a positive control for activation marker induction (thick lines). Binding of CD69 and CD25 mAbs to β7 I-tsNP- and naked siRNA-treated samples were as low as background and the differences are hardly visible. (B) Expression of IFN responsive genes (interferon-β; 2′, 5′-oligoadenylate synthetase, OAS1; or Stat-1) relative to GAPDH as analyzed by quantitative RT-PCR in mouse splenocytes treated with as much as 1 μM luciferase-siRNA delivered as indicated. Poly (I:C) was used as a positive control to induce interferon responses.

FIG. 11. β7 I-tsNP induces gene silencing in human peripheral blood mononuclear cells (PBMC). Note that as FIB504 binds to not only mouse but also human β7 integrins, β7 I-tsNP also proved capable of inducing potent siRNA-mediated silencing of Ku70 in human PBMC. (A). FACS histograms showing binding of β7 I-tsNP (thick line) and IgG sNP (thin line) to human PBMC. (B) Gene silencing in PMBC by Ku70-siRNA delivery with β7 I-tsNP. FACS histograms are shown for PBMC mock treated (dashed line) or treated for 72 h with 1,000 pmol Ku70-siRNA delivered with β7 I-tsNP (thick line) or IgG-sNP (thin line).

FIG. 12. Impact of CyD1-knockdown on cytokine mRNA expression studied under conditions impermissive for substantial cell proliferation. (A & B) Splenocytes were treated with siRNAs (1,000 pmol) delivered as indicated for 12 h in the presence of PMA/ionomycin stimulation. (A) mRNA levels for CyD1 and cytokines were measured by quantitative RT-PCR and normalized to the mRNA expression of GAPDH. (B) Cellular proliferation was measured by [3H]-thymidine incorporation. [3H]thymidine was add at time 0 and incorporated for 12 h. (A & B) Data are expressed as the mean±SEM of three independent experiments. p<0.05*, 0.01† v.s. mock-treated activated cells

FIG. 13. Effects of D-type cyclin-knockdowns on cytokine mRNA expression. (A-F) Cyclin D1 (CyD1 in A & B), Cyclin D2 (CyD2 in C & D), and Cyclin D3 (CyD3 in E & F) were studied in a TK-1 cell line under conditions impermissive for substantial cell proliferation. PMA/ionomycin-stimulated TK-1 cells were treated for 12 h with 1,000 pmol siRNA delivered via β7 I-tsNP or IgG-sNP, or nothing. mRNA levels for cyclins and cytokines were measure by quantitative RT-PCR and normalized to the mRNA expression of GAPDH (A, C, E). [3H]thymidine was added at time 0 and allowed to be incorporated for 12 h (B, D, F). Note that CyD1-knockdown selectively suppressed agonist-upregulated Th1-cytokine mRNA, whereas neither CyD2- nor CyD3-knockdown affected Th1 and Th2 cytokines.

FIG. 14. DSS-induced colitis score. The severity of DSS-induced colitis was histologically graded as previously described (11). †p<0.01.

FIG. 15. Blockade of β7 integrin-MAdCAM-1 interaction by β7 I-tsNP. Because the β7 antibody FIB504 used for generating β7 I-tsNP was previously characterized as a function-blocking antibody (16), the possibility was examined that β7 I-tsNP retained the capacity to directly block any adhesive interaction with MAdCAM-1. Cell adhesion assays using Mn2+- or PMA-stimulated splenocytes showed that β7 I-tsNP interfered with adhesive interactions to MAdCAM-1. (Eun Jeong, Dan, How much β7 I-tsNP and FIB504 did you use?) This result may at least partly account for its mild anti-colitis effect independent of cyclin D1-knockdown (i.e., β7 I-tsNP entrapping irrelevant luciferase siRNA mildly blocked the body weight loss at day 9 in FIG. 4A). Thus, β7 I-tsNP might act synergistically, both through cyclin D1-knockdown and via perturbation of β7 integrin-MAdCAM-1 interactions.

FIG. 16. Hyaluronan-nanoparticles (sNP) entrapping CyD1-siRNA showed no protective effects in DSS-induced colitis. (A & B) To study the possibility that sNP might be sufficient to deliver CyD1-siRNA to CD44high activated leukocytes and/or that the presence of hyaluronan might have any anti-inflammatory effects to ameliorate colitis, 2.5 mg/kg CyD1-siRNA entrapped in sNP, β7 I-tsNP, or IgG-sNP were i.v. injected to mice at days 0, 2, 4, and 6 during the course of DSS-induced colitis. The severity of colitis was monitored by body weight changes over the course of disease (A) and hematocrit values at day 9 (B). (A & B) Data are mean±SEM of 6 mice per group in two independent experiments. Note that in contrast to β7 I-tsNP entrapping CyD1-siRNA, sNP entrapping CyD1-siRNA blocked neither a body weight loss nor a hematocrit reduction in DSS-induced colitis.

### DETAILED DESCRIPTION OF THE INVENTION

The invention relates to the use of cyclin D1 as a target for the treatment and/or prevention of inflammation. It is recognized herein that cyclin D1 inhibition selectively suppresses the production or release of Th1 proinflammatory cytokines, and that such suppression is useful for the treatment of inflammatory disease, including autoimmune diseases characterized by or involving Th1 proinflammatory cytokines. As such, methods described herein can include the measurement of one or more Th1 cytokines or their activities in a subject, and administration of an inhibitor of cyclin D1 expression or activity, particularly where the level of one or more cytokines or their activities is/are increased relative to a standard. Materials, methods and considerations for the therapeutic or prophylactic methods described herein are set out in the following.

### Measurement of Th1 Cytokines

Cytokines, including Th1 cytokines can be measured in various ways, including, but not limited to, e.g., immunoassay for the proteins themselves, or by assays for the expression of mRNA encoding the cytokines, e.g., by RT-PCR. Alternatively, a functional assay that provides a readout of cytokine-mediated activity in a cell-based or other in vitro or in vivo system can also be used.

Samples to be measured for Th1 cytokines will vary depending upon the situation. Where the effect of a given inhibitor on Th1 cytokine production is being assessed experimentally to evaluate the suitability of a given cyclin D1 inhibitor for therapeutic use, the sample can be, for example, cell culture medium or some fraction thereof, or the cultured cells themselves of some fraction thereof. Where the impact of cyclin D1 inhibition is being monitored in vivo, the sample can include, for example, but without limitation, blood, serum, lymphocytes, or a tissue sample from affected tissue.

Immunoassays for cytokines are well known to those of skill in the art and are commercially available from an array of sources. For example, Linco sells a multiplex immunoassay kit (LINCOPLEX™, Linco, St. Charles, Mo., USA) designed to simultaneously identify 8 cytokines, including Th1 cytokines in a single 25 μl sample. See, e.g., Jacob et al., 2003, Mediators Inflamm. 12: 309-313.

As noted, RT-PCR can also be used to measure cytokine production. The skilled artisan can readily prepare primers effective to amplify any one of the inflammatory cytokine mRNAs, including Th1 cytokines. A panel of primers and reagents to identify 84 different human inflammatory cytokines and receptors by real-time PCR is also available from SuperArray, Inc. (“RT² PROFILER™ PCR Array Human Inflammatory Cytokines and Receptors, catalog No. PAHS-011; SuperArray, Inc., Frederick, Md., USA).

In some instances, it can be advantageous to compare cytokine or cytokine mRNA levels to levels in a standard. Standards can include, for example, control samples assayed in parallel to samples from sources, e.g., other individuals or cell samples known to be normal or not affected by an inflammatory or autoimmune disease or disorder. Alternatively, a standard can be an amount or concentration of cytokine understood by the skilled clinician to be characteristic of a healthy individual.

### Agents to Inhibit Cyclin D1:

Any of a number of different approaches can be taken to inhibit cyclin D1 expression or activity. Among these are small molecules that either directly bind to cyclin D1 and inhibit its function or that inhibit or otherwise interfere with the expression of cyclin D1. Also among available approaches, antibodies or RNA interference can be used to inhibit the function and/or expression of cyclin D1.

Small Molecule or Chemical Inhibitors: Small molecule inhibitors of cyclin D1 activity are known in the art. For example, the histone deacetylase inhibitor trichostatin A downregulates cyclin D1 transcription by interfering with NF-κB p65 binding to DNA (Hu & Colburn, 2005, Mol. Cancer Res. 3: 100-109), and the fumagillol derivative TNP-470 inhibits cyclin D1 mRNA expression, but not c-myc mRNA expression (Hori et al., 1994, Biochem. Biophys Res. Commun. 204: 1067-1073).

Pharmaceutically acceptable salts or esters of any small molecule inhibitor of cyclin D1 that retain cyclin D1 inhibitory activity (i.e., retains at least 80% of the activity of the free acid form) are specifically contemplated for use in the methods described herein.

Antibody Inhibitors of Cyclin D1: Antibodies that specifically bind cyclin D1 can be used for the inhibition of the factor in vivo. Antibodies to cyclin D1 are commercially available and can be raised by one of skill in the art using well known methods. The cyclin D1 inhibitory activity of a given antibody, or, for that matter, any cyclin D1 inhibitor, can be assessed using methods known in the art or described herein—to avoid doubt, an antibody that inhibits cyclin D1 will inhibit agonist-enhanced expression of Th1 cytokines in CD3/Cd28- or PMA/ionomycin-stimulated splenocytes or TH-1 cells.

Antibody inhibitors of cyclin D1 can include polyclonal and monoclonal antibodies and antigen-binding derivatives or fragments thereof. Well known antigen binding fragments include, for example, single domain antibodies (dAbs; which consist essentially of single V_{L} or V_{H} antibody domains), Fv fragment, including single chain Fv fragment (scFv), Fab fragment, and F(ab′)₂ fragment. Methods for the construction of such antibody molecules are well known in the art.

Nucleic Acid Inhibitors of cyclin D1 Expression: A powerful approach for inhibiting the expression of selected target polypeptides is RNA interference or RNAi. RNAi uses small interfering RNA (siRNA) duplexes that target the messenger RNA encoding the target polypeptide for selective degradation. siRNA-dependent post-transcriptional silencing of gene expression involves cleaving the target messenger RNA molecule at a site guided by the siRNA.

“RNA interference (RNAi)” is an evolutionally conserved process whereby the expression or introduction of RNA of a sequence that is identical or highly similar to a target gene results in the sequence specific degradation or specific post-transcriptional gene silencing (PTGS) of messenger RNA (mRNA) transcribed from that targeted gene (see Coburn, G. and Cullen, B. (2002) J. of Virology 76(18):9225), thereby inhibiting expression of the target gene. In one embodiment, the RNA is double stranded RNA (dsRNA). This process has been described in plants, invertebrates, and mammalian cells. In nature, RNAi is initiated by the dsRNA-specific endonuclease Dicer, which promotes processive cleavage of long dsRNA into double-stranded fragments termed siRNAs. siRNAs are incorporated into a protein complex (termed “RNA induced silencing complex,” or “RISC”) that recognizes and cleaves target mRNAs. RNAi can also be initiated by introducing nucleic acid molecules, e.g., synthetic siRNAs or RNA interfering agents, to inhibit or silence the expression of target genes. As used herein, “inhibition of target gene expression” includes any decrease in expression or protein activity or level of the target gene or protein encoded by the target gene as compared to a situation wherein no RNA interference has been induced. The decrease will be of at least 10%, 20%, 30%, 40%, 50%, 60%, 70%, 80%, 90%, 95% or 99% or more as compared to the expression of a target gene or the activity or level of the protein encoded by a target gene which has not been targeted by an RNA interfering agent.

The terms “RNA interference” and “RNA interfering agent” as they are used herein are intended to encompass those forms of gene silencing mediated by double-stranded RNA, regardless of whether the RNA interfering agent comprises an siRNA, miRNA, shRNA or other double-stranded RNA molecule.

“Short interfering RNA” (siRNA), also referred to herein as “small interfering RNA” is defined as an RNA agent which functions to inhibit expression of a target gene, e.g., by RNAi. An siRNA may be chemically synthesized, may be produced by in vitro transcription, or may be produced within a host cell. In one embodiment, siRNA is a double stranded RNA (dsRNA) molecule of about 15 to about 40 nucleotides in length, preferably about 15 to about 28 nucleotides, more preferably about 19 to about 25 nucleotides in length, and more preferably about 19, 20, 21, 22, or 23 nucleotides in length, and may contain a 3′ and/or 5′ overhang on each strand having a length of about 0, 1, 2, 3, 4, or 5 nucleotides. The length of the overhang is independent between the two strands, i.e., the length of the overhang on one strand is not dependent on the length of the overhang on the second strand. Preferably the siRNA is capable of promoting RNA interference through degradation or specific post-transcriptional gene silencing (PTGS) of the target messenger RNA (mRNA).

siRNAs also include small hairpin (also called stem loop) RNAs (shRNAs). In one embodiment, these shRNAs are composed of a short (e.g., about 19 to about 25 nucleotide) antisense strand, followed by a nucleotide loop of about 5 to about 9 nucleotides, and the analogous sense strand. Alternatively, the sense strand may precede the nucleotide loop structure and the antisense strand may follow. These shRNAs may be contained in plasmids, retroviruses, and lentiviruses and expressed from, for example, the pol III U6 promoter, or another promoter (see, e.g., Stewart, et al. (2003) RNA April; 9(4):493-501, incorporated by reference herein in its entirety).

The target gene or sequence of the RNA interfering agent may be a cellular gene or genomic sequence, e.g. the cyclin D1 sequence. An siRNA may be substantially homologous to the target gene or genomic sequence, or a fragment thereof. As used in this context, the term “homologous” is defined as being substantially identical, sufficiently complementary, or similar to the target mRNA, or a fragment thereof, to effect RNA interference of the target. In addition to native RNA molecules, RNA suitable for inhibiting or interfering with the expression of a target sequence include RNA derivatives and analogs. Preferably, the siRNA is identical to its target.

The siRNA preferably targets only one sequence. Each of the RNA interfering agents, such as siRNAs, can be screened for potential off-target effects by, for example, expression profiling. Such methods are known to one skilled in the art and are described, for example, in Jackson et al. Nature Biotechnology 6:635-637, 2003. In addition to expression profiling, one may also screen the potential target sequences for similar sequences in the sequence databases to identify potential sequences which may have off-target effects. For example, according to Jackson et al. (Id.) 15, or perhaps as few as 11 contiguous nucleotides, of sequence identity are sufficient to direct silencing of non-targeted transcripts. Therefore, one may initially screen the proposed siRNAs to avoid potential off-target silencing using the sequence identity analysis by any known sequence comparison methods, such as BLAST.

siRNA sequences are chosen to maximize the uptake of the antisense (guide) strand of the siRNA into RISC and thereby maximize the ability of RISC to target human GGT mRNA for degradation. This can be accomplished by scanning for sequences that have the lowest free energy of binding at the 5′-terminus of the antisense strand. The lower free energy leads to an enhancement of the unwinding of the 5′-end of the antisense strand of the siRNA duplex, thereby ensuring that the antisense strand will be taken up by RISC and direct the sequence-specific cleavage of the human cyclin D1 mRNA.

siRNA molecules need not be limited to those molecules containing only RNA, but, for example, further encompasses chemically modified nucleotides and non-nucleotides, and also include molecules wherein a ribose sugar molecule is substituted for another sugar molecule or a molecule which performs a similar function. Moreover, a non-natural linkage between nucleotide residues can be used, such as a phosphorothioate linkage. The RNA strand can be derivatized with a reactive functional group of a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatizes with a variety of groups.

Other useful RNA derivatives incorporate nucleotides having modified carbohydrate moieties, such as 2′O-alkylated residues or 2′-O-methyl ribosyl derivatives and 2′-O-fluoro ribosyl derivatives. The RNA bases may also be modified. Any modified base useful for inhibiting or interfering with the expression of a target sequence may be used. For example, halogenated bases, such as 5-bromouracil and 5-iodouracil can be incorporated. The bases may also be alkylated, for example, 7-methylguanosine can be incorporated in place of a guanosine residue. Non-natural bases that yield successful inhibition can also be incorporated.

The most preferred siRNA modifications include 2′-deoxy-2′-fluorouridine or locked nucleic acid (LAN) nucleotides and RNA duplexes containing either phosphodiester or varying numbers of phosphorothioate linkages. Such modifications are known to one skilled in the art and are described, for example, in Braasch et al., Biochemistry, 42: 7967-7975, 2003. Most of the useful modifications to the siRNA molecules can be introduced using chemistries established for antisense oligonucleotide technology. Preferably, the modifications involve minimal 2′-O-methyl modification, preferably excluding such modification. Modifications also preferably exclude modifications of the free 5′-hydroxyl groups of the siRNA.

The Examples herein provide specific examples of siRNA molecules that effectively target cyclin D1 mRNA.

In a preferred embodiment, the siRNA or modified siRNA is delivered or administered in a pharmaceutically acceptable carrier. Additional carrier agents, such as liposomes, can be added to the pharmaceutically acceptable carrier.

In another embodiment, the siRNA is delivered by delivering a vector encoding small hairpin RNA (shRNA) in a pharmaceutically acceptable carrier to the cells in an organ of an individual. The shRNA is converted by the cells after transcription into siRNA capable of targeting, for example, cyclin D1. In one embodiment, the vector is a regulatable vector, such as tetracycline inducible vector. Methods described, for example, in Wang et al. Proc. Natl. Acad. Sci. 100: 5103-5106, using pTet-On vectors (BD Biosciences Clontech, Palo Alto, Calif.) can be used.

In one embodiment, the RNA interfering agents used in the methods described herein are taken up actively by cells in vivo following intravenous injection, e.g., hydrodynamic injection, without the use of a vector, illustrating efficient in vivo delivery of the RNA interfering agents.

One method to deliver the siRNAs is catheterization of the blood supply vessel of the target organ.

Other strategies for delivery of the RNA interfering agents, e.g., the siRNAs or shRNAs used in the methods of the invention, may also be employed, such as, for example, delivery by a vector, e.g., a plasmid or viral vector, e.g., a lentiviral vector. Such vectors can be used as described, for example, in Xiao-Feng Qin et al. Proc. Natl. Acad. Sci. U.S.A., 100: 183-188. Other delivery methods include delivery of the RNA interfering agents, e.g., the siRNAs or shRNAs of the invention, using a basic peptide by conjugating or mixing the RNA interfering agent with a basic peptide, e.g., a fragment of a TAT peptide, mixing with cationic lipids or formulating into particles.

The RNA interfering agents, e.g., the siRNAs targeting cyclin D1 mRNA, may be delivered singly, or in combination with other RNA interfering agents, e.g., siRNAs, such as, for example siRNAs directed to other cellular genes. Cyclin D1 siRNAs may also be administered in combination with other pharmaceutical agents which are used to treat or prevent diseases or disorders associated with oxidative stress, especially respiratory diseases, and more especially asthma.

Synthetic siRNA molecules, including shRNA molecules, can be obtained using a number of techniques known to those of skill in the art. For example, the siRNA molecule can be chemically synthesized or recombinantly produced using methods known in the art, such as using appropriately protected ribonucleoside phosphoramidites and a conventional DNA/RNA synthesizer (see, e.g., Elbashir, S. M. et al. (2001) Nature 411:494-498; Elbashir, S. M., W. Lendeckel and T. Tuschl (2001) Genes & Development 15:188-200; Harborth, J. et al. (2001) J. Cell Science 114:4557-4565; Masters, J. R. et al. (2001) Proc. Natl. Acad. Sci., USA 98:8012-8017; and Tuschl, T. et al. (1999) Genes & Development 13:3191-3197). Alternatively, several commercial RNA synthesis suppliers are available including, but not limited to, Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). As such, siRNA molecules are not overly difficult to synthesize and are readily provided in a quality suitable for RNAi. In addition, dsRNAs can be expressed as stem loop structures encoded by plasmid vectors, retroviruses and lentiviruses (Paddison, P. J. et al. (2002) Genes Dev. 16:948-958; McManus, M. T. et al. (2002) RNA 8:842-850; Paul, C. P. et al. (2002) Nat. Biotechnol. 20:505-508; Miyagishi, M. et al. (2002) Nat. Biotechnol. 20:497-500; Sui, G. et al. (2002) Proc. Natl. Acad. Sci., USA 99:5515-5520; Brummelkamp, T. et al. (2002) Cancer Cell 2:243; Lee, N. S., et al. (2002) Nat. Biotechnol. 20:500-505; Yu, J. Y., et al. (2002) Proc. Natl. Acad. Sci., USA 99:6047-6052; Zeng, Y., et al. (2002) Mol. Cell 9:1327-1333; Rubinson, D. A., et al. (2003) Nat. Genet. 33:401-406; Stewart, S. A., et al. (2003) RNA 9:493-501). These vectors generally have a polIII promoter upstream of the dsRNA and can express sense and antisense RNA strands separately and/or as a hairpin structures. Within cells, Dicer processes the short hairpin RNA (shRNA) into effective siRNA.

The targeted region of the siRNA molecule of the present invention can be selected from a given target gene sequence, e.g., a cyclin D1 coding sequence, beginning from about 25 to 50 nucleotides, from about 50 to 75 nucleotides, or from about 75 to 100 nucleotides downstream of the start codon. Nucleotide sequences may contain 5′ or 3′ UTRs and regions nearby the start codon. One method of designing a siRNA molecule of the present invention involves identifying the 23 nucleotide sequence motif AA(N19)TT (where N can be any nucleotide) and selecting hits with at least 25%, 30%; 35%, 40%, 45%, 50%, 55%, 60%, 65%, 70% or 75% G/C content. The “TT” portion of the sequence is optional. Alternatively, if no such sequence is found, the search may be extended using the motif NA(N21), where N can be any nucleotide. In this situation, the 3′ end of the sense siRNA may be converted to TT to allow for the generation of a symmetric duplex with respect to the sequence composition of the sense and antisense 3′ overhangs. The antisense siRNA molecule may then be synthesized as the complement to nucleotide positions 1 to 21 of the 23 nucleotide sequence motif. The use of symmetric 3′ TT overhangs may be advantageous to ensure that the small interfering ribonucleoprotein particles (siRNPs) are formed with approximately equal ratios of sense and antisense target RNA-cleaving siRNPs (Elbashir et al. (2001) supra and Elbashir et al. 2001 supra). Analysis of sequence databases, including but not limited to the NCBI, BLAST, Derwent and GenSeq as well as commercially available oligosynthesis companies such as Oligoengine®, may also be used to select siRNA sequences against EST libraries to ensure that only one gene is targeted.

Delivery of RNA Interfering Agents: Methods of delivering RNA interfering agents, e.g., an siRNA, or vectors containing an RNA interfering agent, to the target cells, e.g., lymphocytes or other desired target cells, for uptake include injection of a composition containing the RNA interfering agent, e.g., an siRNA, or directly contacting the cell, e.g., a lymphocyte, with a composition comprising an RNA interfering agent, e.g., an siRNA. In another embodiment, RNA interfering agents, e.g., an siRNA may be injected directly into any blood vessel, such as vein, artery, venule or arteriole, via, e.g., hydrodynamic injection or catheterization. Administration may be by a single injection or by two or more injections. The RNA interfering agent is delivered in a pharmaceutically acceptable carrier. One or more RNA interfering agents may be used simultaneously.

In one preferred embodiment, only one siRNA that targets human cyclin D1 is used.

In one embodiment, specific cells are targeted with RNA interference, limiting potential side effects of RNA interference caused by non-specific targeting of RNA interference. The method can use, for example, a complex or a fusion molecule comprising a cell targeting moiety and an RNA interference binding moiety that is used to deliver RNA interference effectively into cells. For example, an antibody-protamine fusion protein when mixed with siRNA, binds siRNA and selectively delivers the siRNA into cells expressing an antigen recognized by the antibody, resulting in silencing of gene expression only in those cells that express the antigen. The siRNA or RNA interference-inducing molecule binding moiety is a protein or a nucleic acid binding domain or fragment of a protein, and the binding moiety is fused to a portion of the targeting moiety. The location of the targeting moiety can be either in the carboxyl-terminal or amino-terminal end of the construct or in the middle of the fusion protein. Hyaluronan-coated nanoliposomes can be used for delivery; the preparation of hyaluronan-coated liposomes with antibody targeting moieties is described, e.g., in WO 2007/127272, which is incorporated herein by reference. Details of targeting of lymphocytes using particularly effective integrin-binding stabilized nanoparticles comprising siRNA specific for cyclin D1 are provided in the Examples herein.

A viral-mediated delivery mechanism can also be employed to deliver siRNAs to cells in vitro and in vivo as described in Xia, H. et al. (2002) Nat Biotechnol 20(10):1006). Plasmid- or viral-mediated delivery mechanisms of shRNA may also be employed to deliver shRNAs to cells in vitro and in vivo as described in Rubinson, D. A., et al. ((2003) Nat. Genet. 33:401-406) and Stewart, S. A., et al. ((2003) RNA 9:493-501).

The RNA interfering agents, e.g., the siRNAs or shRNAs, can be introduced along with components that perform one or more of the following activities: enhance uptake of the RNA interfering agents, e.g., siRNA, by the cell, e.g., lymphocytes or other cells, inhibit annealing of single strands, stabilize single strands, or otherwise facilitate delivery to the target cell and increase inhibition of the target gene, e.g., cyclin D1.

The dose of the particular RNA interfering agent will be in an amount necessary to effect RNA interference, e.g., post translational gene silencing (PTGS), of the particular target gene, thereby leading to inhibition of target gene expression or inhibition of activity or level of the protein encoded by the target gene.

### Measuring Cyclin D1 Inhibition:

The effectiveness of a given cyclin D1 inhibitor can be monitored in a number of ways. For example, where the inhibitor targets cyclin D1 mRNA, the mRNA itself can be measured, either directly, e.g., as in a Northern blot, or, for example, by RT-PCR. Thus, cultured cells, e.g., splenocytes or other cells, can be treated with one or more doses of an inhibitor, followed by detection of cyclin D1 mRNA by RT-PCR. A reduction in cyclin D1 mRNA, relative to untreated cells or to cells treated with a control agent (e.g., a non-cyclin D1-specific siRNA) would indicate that the inhibitor is functional. Preferably, at least one or more controls is performed, monitoring a transcript other than cyclin D1, in order to evaluate the specificity of the agent.

An alternative approach is to measure cyclin D1 polypeptide directly, e.g., by immunoassay, e.g., by Western blotting, immunoprecipitation, immunofluorescence, or ELISA. Cells cultured with or without the agent are either directly processed for immunofluorescence, or are extracted for proteins, followed by the appropriate assay to detect cyclin D1.

Another alternative approach is to monitor effects of an inhibitor on the downstream activity of cyclin D1, and particularly effects on Th1 cytokine production. Thus, cells, e.g., splenocytes, lymphocytes or a cell line, e.g., TK-1 cells, can be treated with agonist in the presence and absence of a cyclin D1 inhibitor, followed by measurement of Th1 cytokine production as described herein above. The measurement of Th1 and Th2 cytokines following cyclin D1 knockdown in CD3/CD28-treated splenocytes, PMA/ionomycin-treated splenocytes, and PMA/ionomycin-treated TK-1 cells is described in the Examples herein below.

Finally, in vivo models as discussed in the Examples herein can be used to confirm the activity of a given cyclin D1 inhibitor. Levels of cyclin D1 mRNA and/or protein can be measured following administration to an appropriate animal model, or, alternatively, levels of Th1 cytokines can be measured.

### Dosage and Administration:

Cyclin D1 inhibitors are administered in a manner effective to reduce cyclin D1 activity or expression in a tissue undergoing an inflammatory response or in a tissue in which an inflammatory response is wished to be prevented. Delivery methods for RNA interference cyclin D1 inhibitors are described above and in the Examples herein. Other inhibitors, e.g., antibodies or other polypeptide inhibitors can be administered in a manner that preserves the structure and activity of the inhibitory agent.

Cyclin D1 inhibitors can be administered in combination with other anti-inflammatory agents if so desired. The cyclin D1 inhibitor agent plus second anti-inflammatory agent combination can be administered as an admixture of the agents, or the agents can be administered separately to the individual. In general, the cyclin D1 inhibitory agent and the other therapeutic agent do not have to be administered in the same pharmaceutical composition, and may, because of different physical and chemical characteristics, have to be administered by different routes. For example, the cyclin D inhibitory agent may be administered orally to generate and maintain good blood levels thereof, while the other agent may be administered by inhalation, or vice versa. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

Thus, in accordance with experience and knowledge, the practicing physician can modify each protocol for the administration of a component of the treatment according to the individual patient's needs, as the treatment proceeds.

Pharmaceutical Compositions: Inert, pharmaceutically acceptable carriers or excipients used for preparing pharmaceutical compositions of the cyclin D1 inhibitors described herein can be either solid or liquid. Solid preparations include powders, tablets, dispersible granules, capsules, cachets and suppositories. The powders and tablets may comprise from about 5 to about 70% active ingredient. Suitable solid carriers are known in the art, e.g., magnesium carbonate, magnesium stearate, talc, sugar, and/or lactose. Tablets, powders, cachets and capsules can be used as solid dosage forms suitable for oral administration.

For preparing suppositories, a low melting wax such as a mixture of fatty acid glycerides or cocoa butter is first melted, and the active ingredient is dispersed homogeneously therein as by stirring. The molten homogeneous mixture is then poured into conveniently sized molds, allowed to cool and thereby solidify.

Liquid preparations include solutions, suspensions and emulsions. As an example can be mentioned water or water-propylene glycol solutions for parenteral injection, e.g., intravenous injection. Liquid preparations can also include solutions for intranasal administration. Aerosol preparations suitable for inhalation can include solutions and solids in powder form, which can be in combination with a pharmaceutically acceptable carrier, such as an inert compressed gas.

Also included are solid preparations which are intended for conversion, shortly before use, to liquid preparations for either oral or parenteral administration. Such liquid forms include solutions, suspensions and emulsions.

The cyclin D1 inhibitory agents described herein can also be deliverable transdermally. The transdermal compositions can take the form of creams, lotions, aerosols and/or emulsions and can be included in a transdermal patch of the matrix or reservoir type as are conventional in the art for this purpose.

The suitability of a particular route of administration will depend in part on the pharmaceutical composition (e.g., whether it can be administered orally without decomposing prior to entering the blood stream). Controlled release systems known to those skilled in the art can be used where appropriate.

Preferably, the pharmaceutical preparation is in unit dosage form. In such form, the preparation is subdivided into unit doses containing appropriate quantities of the active component, e.g., an effective amount to achieve the desired purpose.

The actual dosage employed may be varied depending upon the requirements of the patient and the severity of the condition being treated. Determination of the proper dosage for a particular situation is within the skill of the art. Generally, treatment is initiated with smaller dosages which are less than the optimum dose of the compound. Thereafter, the dosage is increased by small amounts until the optimum effect under the circumstances is reached. For convenience, the total daily dosage may be divided and administered in portions during the day if desired.

The amount and frequency of administration of the cyclin D1 inhibitory agents will be regulated according to the judgment of the attending clinician (physician) considering such factors as age, condition and size of the patient as well as severity of the disease being treated. Amounts needed to achieve the desired effect, i.e., a “therapeutically effective dose” will vary with these and other factors known to the ordinarily skilled practitioner, but generally range from 0.001 to 5.0 mg of inhibitory agent per kilogram of body weight, with doses of 0.05 to 2.0 mg/kg/dose being more commonly used. For prophylactic or maintenance applications, compositions containing the cyclin D1 inhibitory agent can also be administered in similar or slightly lower dosages relative to therapeutic dosages, and often with lower frequency (illustrative examples include, every other day or even weekly or monthly for a maintenance or preventative regimen, as opposed to, for example, every day for a therapeutic regimen). The frequency of dosages for either therapeutic or maintenance/prophylactic uses will also depend, for example, on the in vivo half-life of the cyclin D1 inhibitor used. Thus, more frequent dosing is appropriate where the half-life is shorter, and vice versa. One of skill in the art can measure the in vivo half-life for a given cyclin D1 inhibitor. Where appropriate, and especially, for example, when the agent will be administered systemically (e.g., intravenously or other systemic route), it is specifically contemplated that cyclin D1 inhibitors can be coupled to agents that increase the in vivo half-life of the agent. For example, polypeptides or other agents can be coupled to a serum protein, e.g., serum albumin, to increase the half-life of the polypeptide. Targeted delivery of cyclin D1 inhibitors is discussed in detail in the Examples herein.

The cyclin D1 inhibitory agent or treatment can be administered according to therapeutic protocols well known in the art. It will be apparent to those skilled in the art that the administration of a cyclin D1 inhibitory therapy can be varied depending on the disease being treated and the known effects of the agent administered on that disease. Also, in accordance with the knowledge of the skilled clinician, the therapeutic protocols (e.g., dosage amounts and times of administration) can be varied in view of the observed effects of the administered therapeutic agents (e.g., amelioration of symptoms) on the patient, and in view of the observed responses of the disease to the administered therapeutic agents.

### Measuring Efficacy:

The efficacy of treatment of inflammation or autoimmune disease as described herein can be measured in a variety of ways. For example, standard clinical markers of inflammation itself can be measured, e.g., edema, lymphocyte infiltration or other histopathological marker, or inflammatory cytokine levels, among others. A statistically significant change in any such clinically relevant marker is indicative of effective treatment.

Similarly, the effect on inflammatory or autoimmune disease can be determined by tracking one or more symptoms or accepted indicators of disease status for a given disease or disorder. Thus, clinically accepted scales for disease grading known to the ordinarily skilled clinician can be applied to evaluate the efficacy of treatment involving inhibition of cyclin D1. A statistically significant decrease in disease severity as measured by such a scale, or, in the instance where a disease is progressive, a cessation or statistically significant slowing in the worsening of pathological state can indicate effective treatment.

Examples of clinically accepted scales for grading inflammatory disease include, for example, the Ulcerative Colitis Scoring System (UCSS; see, e.g., Nikolaus et al., 2003, Gut 52:1286-1290). As an example, when using the UCSS, an effective response in treatment of UC is determined where there is a decrease of at least 3 points from baseline in the symptoms score, preferably, but not necessarily including the induction of endoscopically confirmed remission.

Rheumatoid arthritis can be measured, for example, by the Rheumatoid Arthritis Severity Scale, or RASS, described by Bardwell et al., 2002, Rheumatology 41: 38-45. Alternatives include the Personal Impact Health Assessment Questionnaire (PI HAQ), described by Hewlett et al., Ann Rheum Dis. 2002 November; 61(11): 986-993, and the Rheumatoid Arthritis Quality of Life scale (see, e.g., J. Rheumatol. 2001; 28:1505-1510).

Multiple sclerosis severity can be measured, for example, on the Kurtzke Expanded Disability Status Scale (EDSS) (see, e.g., Kurtzke, 1983, Neurology 33: 1444-1452) or on the Symptoms of Multiple Sclerosis Scale (SMSS; see, e.g., Arch. Phys. Med. Rehabil. 2006, 87: 832-41).

Psoriasis severity can be scaled, for example, using the National Psoriasis Foundation Psoriasis Score System (NPF-PSS) or the Psoriasis Area Severity Index and Physician's Global Assessment (see, e.g, Gottlieb et al., 2003, J. Drugs Rheum. for a comparison of the two approaches).

Lupus severity can be scored, for example, on the British Isles Lupus Assessment Group (BILAG) score (see, e.g., Gordon et al., 2003, Rheumatology 2003; 42: 1372-1379).

Other autoimmune or inflammatory disorders or diseases can be similarly measured according to clinically accepted scales known to those of skill in the art.

As an alternative or in addition to measurement of clinical stage of disease, the presence or amount of inflammatory cytokines, particularly Th1 cytokines, can be measured to determine efficacy of treatment or prevention. Measurements of, e.g., serum or tissue levels of Th1 cytokines can be performed as described herein above. A statistically significant reduction in the level of one or more of such cytokines is an indicator of effective treatment using an inhibitor of cyclin D1 as described herein. To avoid doubt, a reduction in the level of at least one Th1 cytokine by at least 10%, and preferably at least 20%, 30%, 40%, 50%, 60%, 70%, 80%, 90%, 95%, 97%, 98%, 99% or even 100% (i.e., absence of the cytokine) following treatment as described herein is considered to indicate efficacy.

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages means±1%.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The present invention may be as defined in any one of the following numbered paragraphs.

1. Use of an agent that inhibits cyclin D1 for the preparation of a medicament for the treatment or prevention of inflammation in a subject in need thereof, wherein administering said agent reduces or prevents inflammation in a said subject.

2. Use of an agent that inhibits cyclin D1 for the preparation of a medicament for the treatment or prevention of Th1-mediated inflammation in a subject in need thereof, wherein administering said agent reduces or prevents Th1-mediated inflammation in a said subject.

3. Use of an agent that inhibits cyclin D1 for the preparation of a medicament for the treatment of an autoimmune disease or a disorder characterized by or involving a Th1 inflammatory response in a subject in need thereof, wherein administering said agent to said subject reduces said Th1 inflammatory response.

4. Use of an agent that inhibits cyclin D1 for the treatment or prevention of inflammation in a subject in need thereof, wherein administering said agent reduces or prevents inflammation in a said subject.

5. Use of an agent that inhibits cyclin D1 for the treatment or prevention of Th1-mediated inflammation in a subject in need thereof, wherein administering said agent reduces or prevents Th1-mediated inflammation in a said subject.

6. Use of an agent that inhibits cyclin D1 for the treatment of an autoimmune disease or a disorder characterized by or involving a Th1 inflammatory response in a subject in need thereof, wherein administering said agent to said subject reduces said Th1 inflammatory response.

7. A method for treating or preventing inflammation, the method comprising administering an agent that inhibits cyclin D1 to a subject in need thereof, wherein inflammation is reduced or prevented.

8. A method of selectively inhibiting Th1-mediated inflammation, the method comprising administering an agent that inhibits cyclin D1 to a subject in need thereof, wherein said Th1-mediated inflammation is inhibited.

9. The method of paragraphs 7 or 8 further comprising the step of determining a level of at least one Th1 cytokine in a sample from said individual and comparing said level to a standard, and, if an increased level of at least one said Th1 cytokine is found, said agent is administered to said individual.

10. The method of paragraph 9 or the use of any one of claims 1-6 wherein said administering reduces the expression of a Th1 cytokine.

11. The method of paragraph 9 wherein said Th1 cytokine is selected from the group consisting of TNF-α, IL-2, IL-12, IFN-γ and IL-23.

12. The use of any one of paragraphs 1-6 or the method of any one of paragraphs 7-11 wherein said agent comprises an antibody, a nucleic acid or a small molecule.

13. The use of any one of paragraphs 1-6 or the method of any one of paragraphs 7-12 wherein said agent comprises a nucleic acid.

14. The use of any one of paragraphs 1-6 or the method of any one of paragraphs 7-13 wherein said agent comprises an interfering RNA.

15. The use of any one of paragraphs 1-6 or the method of any one of paragraphs 7-14 wherein said agent comprises a targeting moiety.

16. The method or use of paragraph 15 wherein said targeting moiety targets said agent to a leukocyte.

17. The method or use of paragraph 16 wherein said targeting moiety binds cell surface molecule expressed on a target cell.

18. The method or use of paragraph 17 wherein said targeting moiety binds an integrin molecule.

19. The method or use of any one of paragraphs 15-18 wherein said targeting moiety binds integrin B7.

20. The method or use of paragraph 14 wherein said interfering RNA targets a cyclin D1 mRNA for degradation.

21. A method of treating an autoimmune disease or a disorder characterized by or involving a Th1 inflammatory response in a subject in need thereof, the method comprising administering to said subject an agent that inhibits cyclin D1, wherein said Th1 inflammatory response is reduced.

22. The method of paragraph 21 further comprising the step of determining a level of at least one Th1 cytokine in a sample from said subject, and comparing said level to a standard, and, if an increased level of at least one said Th1 cytokine is found, said agent is administered to said subject.

23. The method of paragraph 22 wherein the step of determining a level of at least one Th1 cytokine comprises determining a level of a cytokine selected from the group consisting of TNF-α, IL-2, IL-12, IFN-γ and IL-23.

24. The method of any one of paragraphs 21-23 or the use of paragraph 3 or paragraph 6 wherein said autoimmune disease or disorder is selected from the group consisting of an inflammatory bowel disease, ulcerative colitis, Crohn's disease, celiac disease, autoimmune hepatitis, chronic rheumatoid arthritis, psoriatic arthritis, insulin-dependent diabetes mellitus, multiple sclerosis, Alzheimer's disease, enterogenic spondyloarthropathies, autoimmune myocarditis, psoriasis, scleroderma, myasthenia gravis, multiple myositis/dermatomyositis, Hashimoto's disease, autoimmune hypocytosis, pure red cell aplasia, aplastic anemia, Sjogren's syndrome, vasculitis syndrome, systemic lupus erythematosus, glomerulonephritis, pulmonary inflammation (e.g., interstitial pneumonia), septic shock and transplant rejection.

25. The method of any one of paragraphs 21-24 wherein said administering reduces the expression of a Th1 cytokine.

26. The method of paragraph 25 wherein said Th1 cytokine is selected from the group consisting of TNF-α, IL-2, IL-12, IFN-γ and IL-23.

27. The method of any one of paragraphs 21-26 wherein said agent comprises an antibody, a nucleic acid or a small molecule.

28. The method of any one of paragraphs 21-27 wherein said agent comprises a nucleic acid.

29. The method of any one of paragraphs 21-28 wherein said agent comprises an interfering RNA.

30. The method of any one of paragraphs 21-29 wherein said agent comprises a targeting moiety.

31. The method of paragraph 30 wherein said targeting moiety targets said agent to a leukocyte.

32. The method of paragraphs 31 or 32 wherein said targeting moiety binds a cell surface molecule expressed on a target cell.

33. The method of any one of paragraphs 30-32 wherein said targeting moiety binds an integrin molecule.

34. The method of any one of paragraphs 30-33 wherein said targeting moiety binds integrin B7.

35. The method of paragraph 29 wherein said interfering RNA targets a cyclin D1 nucleic acid for degradation.

36. The method of paragraph 24 wherein said inflammatory bowel disease is Crohn's disease or ulcerative colitis.

This invention is further illustrated by the following examples which should not be construed as limiting. The contents of all references cited throughout this application, as well as the figures and table are incorporated herein by reference.

### EXAMPLES

RNA interference (RNAi) has emerged as a powerful strategy to suppress gene expression, holding the potential to dramatically accelerate in vivo drug target validation as well as the promise to create novel therapeutic approaches if it can be effectively applied in vivo (1). Cyclin D1 (CyD1) is a key cell-cycle-regulating molecule that governs proliferation of normal and malignant cells (2, 3). In inflammatory bowel diseases, colon-expressed CyD1 is aberrantly unregulated in both epithelial and immune cells (4, 5). Although CyD1 has also been implicated in promoting epithelial colorectal dysplasia and carcinogenesis, it is not clear whether leukocyte-expressed CyD1 contributes directly to the pathogenesis of inflammation and if it might serve as a therapeutic target.

### Example 1

### Methods and Inhibitors of Cyclin D1

Preparation of Integrin Targeted and Stabilized Nanoparticles (1-tsNP).

Hyaluronan (HA) coated nanoliposomes were prepared as described (1). The method is also described in WO 2007/127272, which is incorporated herein by reference. Multilamellar liposomes (MLL), composed of phosphatidylcholine (PC), dipalmitoylphosphatidylethanolamine (DPPE), and cholesterol (Chol) at mole ratios of 3:1:1 (PC:DPPE:Chol), were prepared by a lipid-film method (2). A lipid film was hydrated with 20 mM Hepes-buffered saline pH 7.4 to create MLL. Lipids were obtained from Avanti Polar Lipids, Inc., (Alabaster, Ala.). Lipid mass was measured as previously described (3). Resulting MML were extruded into unilamellar nano-scale liposomes (ULNL) with a Thermobarrel Lipex Extruder™ (Lipex biomembranes Inc., Vancouver, British Columbia, Canada) at room temperature under nitrogen pressures of 300 to 550 psi. The extrusion was carried out in a stepwise manner using progressively decreasing pore-sized membranes (from 1, 0.8, 0.6, 0.4, 0.2, to 0.1 μm) (Nucleopore, Whatman), with 10 cycles per pore-size. ULNL were surface-modified with high molecular weight HA (850 KDa, intrinsic viscosity: 16 dL/g, Genzyme Corp, Cambridge, Mass.), as described (1, 4). Briefly, HA was dissolved in water and pre-activated with EDC, at pH 4.0 for 2 h at 37° C. Resulting activated HA was added to a suspension of DPPE-containing ULNL in 0.1M borate buffer pH of 8.6, and incubated overnight at 37° C., under gentle stirring. Resulting HA-ULNL were separated by centrifugation (1.3×105 g, 4° C., for 1 h) and washed four times. The final HA/lipid ratio was typically 75 μg HA/μmole lipid as assayed by 3H-HA (ARC, Saint Louis, Mich.). HA-modified liposomes were coupled to mAbs using an amine-coupling method. Briefly, 50 μL HA-modified liposomes were incubated with 200 μA of 400 mmol/L 1-(3-dimethylaminopropyl)-3-ethylcarbodimide hydrochloride (EDAC, Sigma-Aldrich, Saint Louis, Mich.) and 200 μL of 100 mmol/L N-hydroxysuccinimide (NHS, Fluka, Sigma-Aldrich, Saint Louis, Mich.) for 20 minutes at room temperature with gentle stirring. Resulting NHS-activated HA-nanoliposomes were mixed with 50 μL mAb (10 mg/mL in HBS, pH 7.4) and incubated for 150 min at room temperature with gentle stirring. Twenty microlitter 1M ethanolamine HCl (pH 8.5) was then added to block reactive residues. I-tsNP and IgG-sNP were purified by using a size exclusion column packed with sepharose CL-4B beads (Sigma-Aldrich, Saint Louis, Mich.) equilibrated with HBS, pH 7.4 to remove unattached mAbs.

Particle suspensions in 0.2 mL aliquots were frozen for 2-4 h at −80° C. and lyophilized for 48 h using an alpha 1-2 LDplus lyophilizer (Christ, Osterode, Germany). Lyophilized samples were rehydrated by adding 0.2 ml DEPC-treated water (Ambion Inc., Austin, Tex.) or DEPC-treated water containing protamine-condensed siRNAs.

Particle diameters and surface charges (zeta potential) were measured using a Malvern Zetasizer nano ZS™ (Malvern Instruments Ltd., Southborough, Mass.). The number of liposomes in a given lipid mass was calculated as previously described (5). 125I-labeled FIB504 and isotype control Rat IgG2a were used to measure the number of mAbs per liposome to assess the coupling efficiency. Iodination of mAbs was carried out using Iodo-Gen iodination reagent (Pierce) according to the manufacturer's protocol.

Preparation of siRNAs.

siRNAs from Dharmacon were deprotected and annealed according to the manufacturer's instructions. Four Ku70-siRNAs were used in an equimolar ratio as previously described (6). Cyclin-D1-siRNAs sequences were as follows: ACACCAAUCUCCUCAACGAUU (sense #1); 5′-PUCGUUGAGGAGAUUGGUGUUU (antisense #1); GCAUGUUCGUGGCCUCUAAUU (sense #2); 5′-PUUAGAGGCCACGAACAUGCUU (antisense #2); GCCGAGAAGUUGUGCACUUUU (sense #3); 5′-PAGAUGCACAACUUCUCGGCUU (antisense #3); GCACUUUCUUUCCAGAGUCUU (sense #4); 5′-PGACUCUGGAAAGAAAGUGCUU (antisense #4). Cyclin-D2 siRNA sequences were as follows: GAACUGGUAGUGUUGGGUAUU (sense strand) and 5′-PUACCCAACACUACCAGUUCUU (antisense strand). Cyclin-D3 siRNA sequences were as follows: CUAGAACAAUCCAUGCUAUUU (sense strand) and 5% PAUAGCAUGGAUUGUUCUAGUU (antisense strand). Unless otherwise mentioned, cyclin-D1-siRNAs were used as a cocktail of #1-4 in an equimolar ratio.

siRNA Entrapment in Nanoparticles.

siRNAs were mixed with full-length recombinant protamine (Abnova, Taipei City, Taiwan) in a 1:5 (siRNA:protein) molar ratio, in DEPC-treated water (Ambion Inc., Austin, Tex.) and were pre-incubated for 30 min at RT to form a complex (6). For entrapment, lyophilized nanoparticles (i.e., β7 I-tsNP, IgG-sNP, or sNP; 1˜2.5 mg lipids) were rehydrated by adding 0.2 ml DEPC-treated water containing protamine-condensed siRNAs (1,000˜3,500 pmol). The entrapment procedure was performed immediately before use. Concentrations of siRNAs and percent entrapment were determined by a Quant-iT™ RiboGreen™ RNA assay (Molecular Probes, (Invitrogen) as described (7).

In Vitro Transfection of siRNAs Using β7 I-tsNP.

Splenocytes or TK-1 cells that had been pre-cultured overnight at 37° C., 5% CO2 in 24-well microtiter plates (2.5×105 cells in 200 μl media/well) were given aliquots (50 μl/well) of β7 I-tsNP entrapping siRNAs or appropriate controls in the presence or absence of stimulation with immobilized CD3/CD28 mAbs (20 μg/ml each) or 2.5 ng/ml PMA plus 1 μg/ml iomomycin. Cells were cultured for 6 to 72 h at 37° C., 5% CO2 and subjected to flow cytometry and/or real time RT-PCR analyses.

In some experiments, TK-1 cells, pretreated for 12 h with 2.5 μg/ml aphidicolin to arrest cell cycle, were treated for another 12 h with β7 I-tsNP entrapping siRNAs or appropriate controls in the presence of 2.5 μg/ml aphidicolin and in the presence or absence of PMA/iomomycin.

### Serum and RNase Stability.

Naked Ku70-siRNAs or Ku70-siRNAs entrapped in β7 I-tsNP were incubated with 50% FCS or RNase A (20 ng/mL) for the indicated duration (0, 30, 60, and 120 min). Treated naked siRNAs were transfected to TK-1 cells using Amaxa™ nucleofection according to the manufacture's instructions. Treated β7 I-tsNP-entrapped Ku70-siRNAs were transfected to TK-1 cells as described above.

### Cell Proliferation.

3H-thymidine (1 μCi) was added for 16 h to treated lymphoid cells (5×104) in microtiter wells. Cells were harvested and analyzed by scintillation counting using a Top Count microplate reader (Packard).

### Interferon Assay.

Splenocytes (1×106 cells/ml) were mock treated or treated for 48 hrs with β7 I-tsNP entrapping 1,000 pmol luciferase-siRNA or 5 μg/ml poly (I:C). Expression of IFN or interferon responsive genes was examined by quantitative RT-PCR.

Cell adhesion assay. Cell adhesion to a V-bottom-well plate was studied as previously described (8).

### Quantitative RT-PCR.

Quantitative RT-PCR using a Biorad iCycler was carried out as previously described (9). Primers for mouse GAPDH, STAT1, OAS1, and INF β were used as previously described (9). The following primer pairs were used:

Cyclin D1:
Forward 5′-CTTCCTCTCCAAAATGCCAG-3′
Reverse 5′-AGAGATGGAAGGGGGAAAGA-3′
Cyclin D2:
Forward 5′-CCAAAGGAAGGAGGTAAGGG-3′
Reverse 5′-GCCGGTCACCACTCGG-3′
Cyclin D3:
Forward 5′-TCCTGCCTTCCTCTCCGTAG-3′
Reverse 5′-TCCAGTCACCTCCACGGC-3′
TNF α:
Forward 5′-CCTGTAGCCCACGTCGTAGC-3′,
Reverse 5′-TTGACCTCAGCGCTGAGTTG-3′
IFN-γ:
Forward: 5-TGAACGCTACACACTGCATCTTGG-3
Reverse: 5′-CTCAGGAAGCGGAAAAGGAGTCG-3′
IL-2:
Forward: 5′-TGCAAACAGTGCACCTACTTCAA-3′
Reverse: 5′-CCAAAAGCAACTTTAAATCCATCTG-3′.
IL-12 p40:
Forward: 5′-CTCACATCTGCTGCTCCACAAG-3′;
Reverse: 5′-AATTTGGTGCTTCACACTTCAGG-3′;
IL-10:
Forward: 5′-GGTTGCCAAGCCTTATCGGA-3′;
Reverse: 5′-ACCTGCTCCACTGCCTTGCT-3′:
IL-4:
Forward: 5′-GAATGTACCAGGAGCCATATC-3′
Reverse: 5′-CTCAGTACTACGAGTAATCCA-3′

mRNA expression levels of each transcript were normalized to that of GAPDH as previously described (9).

### Cell Isolation and Flow Cytometry.

Mononuclear cells were isolated from the spleen and gut as previously described (10). Flow cytometry of cell surface antigens was performed as previously described (6). For intracellular staining of cyclin D1 and Ku70, cells were fixed and permeabilized with the Fix-and-Perm Kit™ (Caltag Laboratories, Burlingame, Calif.), stained with 1 μg/ml rabbit anti-mouse cyclin D1 (Santa Cruz Biotechnology, Santa Cruz, Calif.) on ice for 30 min, and counter-stained with FITC-conjugated goat anti-rabbit IgG (Zymed). Detection of Ku70 expression was conducted as previously described (6).

### Image Acquisition and Processing

Confocal imaging was performed using a Biorad Radiance 2000 Laser-scanning confocal system (Hercules, Calif.) incorporating with an Olympus BX50BWI microscope fitted with an Olympus 100× LUMPlanFL 1.0 water-dipping objective. Image acquisition was performed using Laserscan 2000 software and image processing was performed with Openlab 3.1.5 software (Improvision, Lexington, Mass.). (Chris, I need you to revise this part).

Mice and Colitis Model.

Wild-type and β7 integrin knockout mice with a C57BL/6 background were obtained from Charles River Laboratories and maintained in a specific pathogen-free animal facility in the Warren Alpert Building at Harvard Medical School. All animal experiments were approved by the Institutional Review Board of the CBR Institute for Biomedical Research.

Dextran sodium sulphate (DSS)-induced colitis in mice occurred as previously described (10). Briefly, C57BL/6 (Charles River Laboratories) mice were fed for 9 days with 3.5% (wt/vol) DSS (MP Biomedicals, Inc.) in drinking water. Body weight and clinical symptoms were monitored daily. Mice were sacrificed on day 10 and the entire colon was removed from cecum to anus, with colon length measured as a marker of inflammation. Distal colon cross-sections were stained with haematoxylin and eosin for histologic examination. Quantitative histopathologic grading of colitis severity was assessed as previously described (11). Blood was obtained by cardiac puncture. Hematocrit was measured by HEMAVET™ 850 autoanalyzer (Drew Scientific Inc., Dallas, Tex.). Suspensions (200 μl) of nanoparticles entrapping siRNAs were subjected to a sonication in a bath sonicator (Branson 3510) for 5 min, and immediately i.v. injected via tail veins to mice.

### Tissue Distribution Studies and Pharmacokinetic Analysis.

Radiolabeled β7 I-tsNP and IgG sNP were prepared by incorporating the non-exchangeable lipid label 3H-cholesterylhexadecylether (3H-CHE, 5 μCi/mg lipid) as previously described (3). Suspensions (200 μl) of nanoparticles were subjected to sonication in a bath sonicator (Branson 3510) for 5 min, and immediately i.v. injected via tail veins to 8-week-old female C57BL/6 mice (Charles River Laboratories) with or without DSS-induced colitis. Blood was sampled from the retro-orbital vein at 1, 6, and 12 h. Plasma was isolated from whole blood by centrifugation at 3000×g for 5 min. Tissue homogenates 10% (w/v) were prepared in water using a Polytron homogenizer (Brinkman Instruments, Mississauga, Ontario, Canada). Aliquots (200 μl) of tissue homogenate or plasma samples were mixed with 500 μl Solvable™ (Perkin Elmer), and then incubated for 2 h at 60° C. and for 1 h at room temperature for digestion. Digested samples (˜700 μl aliquot) were mixed with 50 μl of 200 mM EDTA and 200 μl of hydrogen peroxide [30% (v/v)], incubated overnight for bleaching, and, following addition of 100 μl of 1 N HCl and 5 ml Ultima Gold, subjected to 3H scintillation counting with a Beckman LS 6500 liquid scintillation counter. Blood correction factors were applied as previously described (3).

### Statistical Analysis.

In vitro data were analyzed using Student's t-test. Differences between treatment groups were evaluated by one-way ANOVA with significance determined by Bonferroni-adjusted t-tests. We used the generalized estimating equations (GEE) approach to model disease scores collected over time and to compare disease severity of control versus treated groups (12).

### Example 2

### RNAi Silencing of Cyclin D1 in Leukocytes In Vitro and In Vivo

RNAi silencing of CyD1 was performed in an experimental model of intestinal inflammation. A major limitation to the use of RNAi in vivo is the effective delivery of small interfering (si)RNAs to the target cells (6, 7). RNAi in leukocytes, a prime target for anti-inflammation, has remained particularly challenging, as they are difficult to transduce with conventional transfection and exhibit diverse distribution patters, often localized deep within tissues, requiring systemic delivery approaches (8). One possibility is the use of integrins, which are an important family of cell-surface adhesion molecules that have potential utility as targets for siRNA delivery (8). Specifically it has been shown that antibody-protamine fusion proteins directed to the leukocyte integrin LFA-1 selectively delivered siRNAs in leukocytes, both in vitro and in vivo (8). However, whether an integrin-directed siRNA delivery approach can induce sufficiently robust silencing in vivo remained to be seen until the experiments described herein were performed.

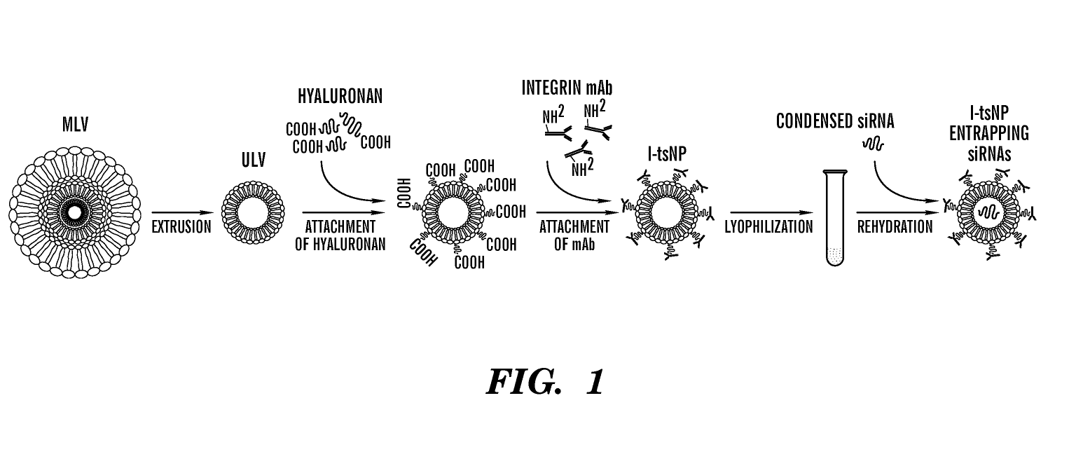

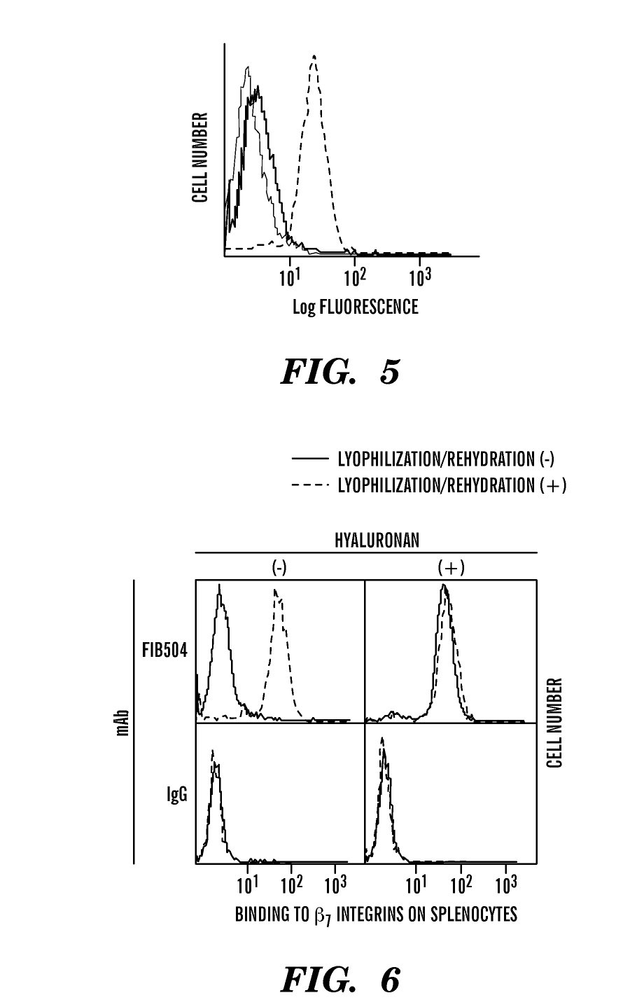

To build on the basic premise of integrin-targeted siRNA delivery, liposome-based β7 integrin-targeted, stabilized nanoparticles β7 I-tsNP) were developed that entrap siRNAs (FIG. 1). This began with nanometer scale (˜80 nm) liposomes, derived specifically from neutral phospholipids allowing the potential toxicity common to cationic lipids and polymers used for systemic siRNA delivery to be circumvented (9). Hyaluronan was then attached to the outer surface of the liposomes, through covalent linkage to dipalmitoylphosphatidylethanolamine. In this way, the particles were stabilized, both during subsequent siRNA entrapment, and during systemic circulation in vivo (10) (FIG. 1). The resulting stabilized nanoparticles (sNP) were successfully rendered the targeting capacity by covalently attaching a monoclonal antibody against the integrins, to hyaluronan (FIG. 5). The antibody FIB504 (11) was selected to direct particles to β7 integrins, which are highly expressed in gut mononuclear leukocytes (12).

β7 I-tsNP were loaded with siRNA cargo by rehydrating lyophilized particles in the presence of condensed siRNAs, thereby achieving ˜80% entrapment efficacy while maintaining the nano-dimensions of particles (Tables S1 and S2). Importantly, β7 I-tsNP showed a measurable increase in their capacity to entrap siRNAs such that I-tsNP carried ˜4,000 siRNA molecules per vehicle (˜100 siRNA molecules per targeting moiety) (Table S1), compared to an integrin-targeted single chain antibody protamine fusion protein, which carried 5 siRNA molecules per vehicle (8). The presence of hyaluronan was critical to maintaining the structural integrity of I-tsNP during a cycle of lyophilization/rehydration (Table 3, FIG. 6).

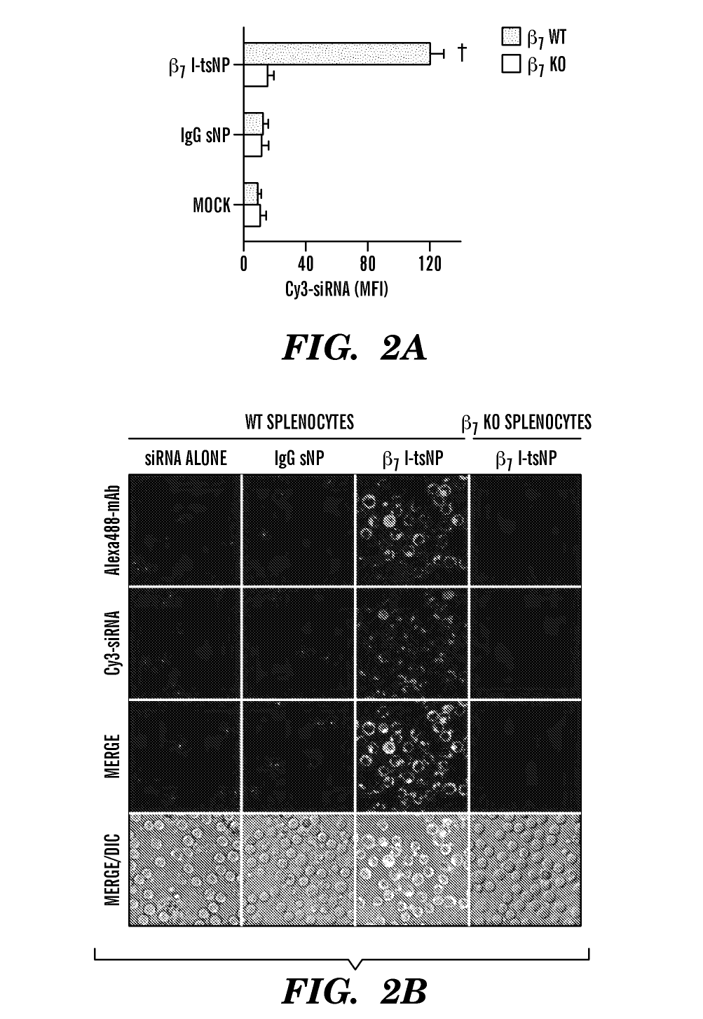

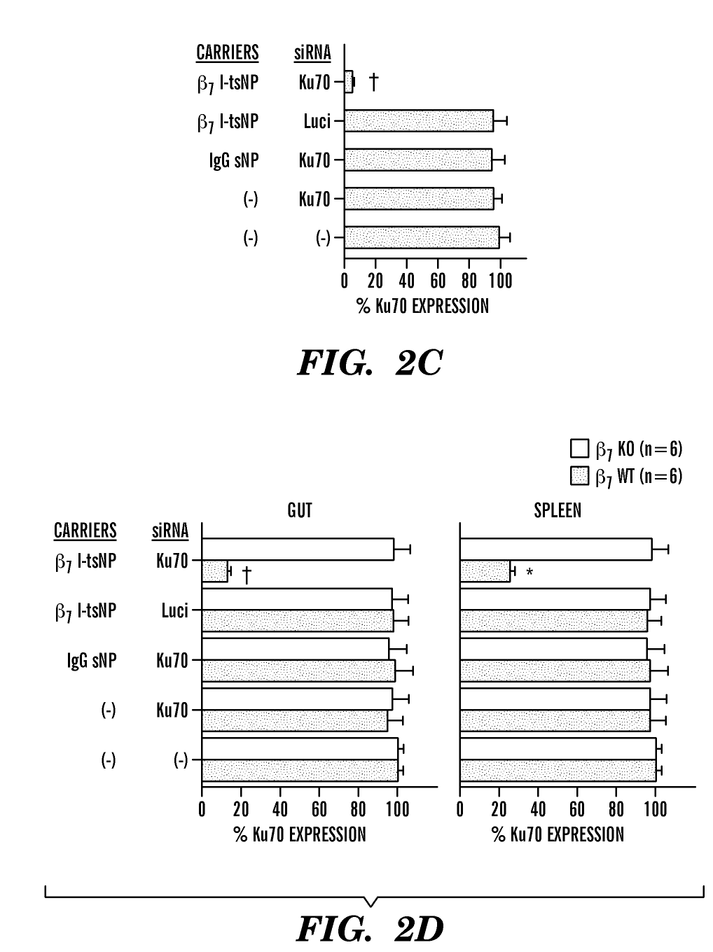

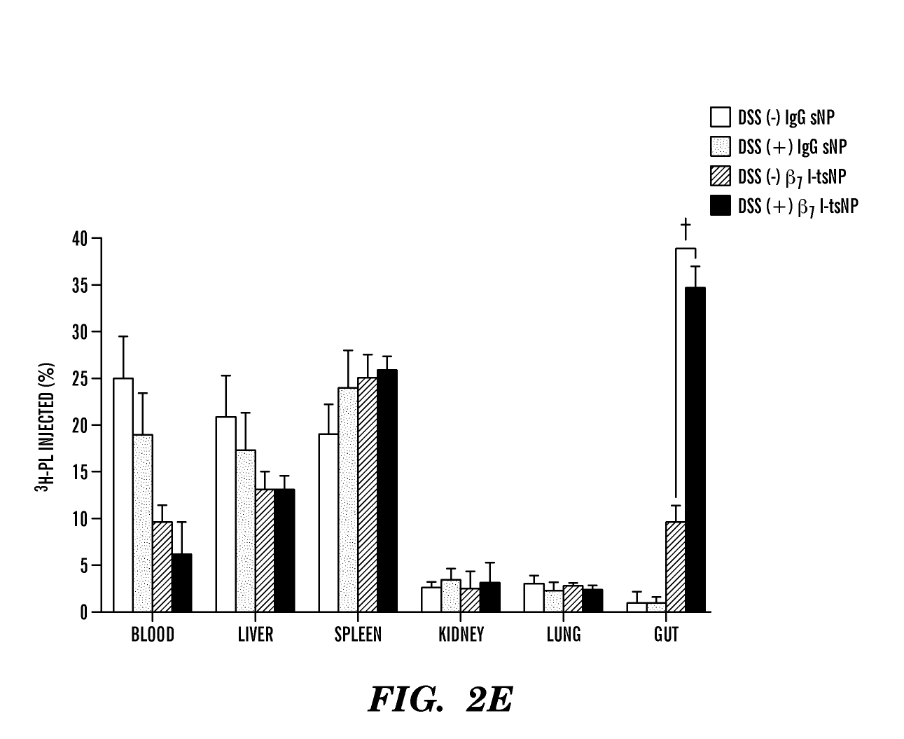

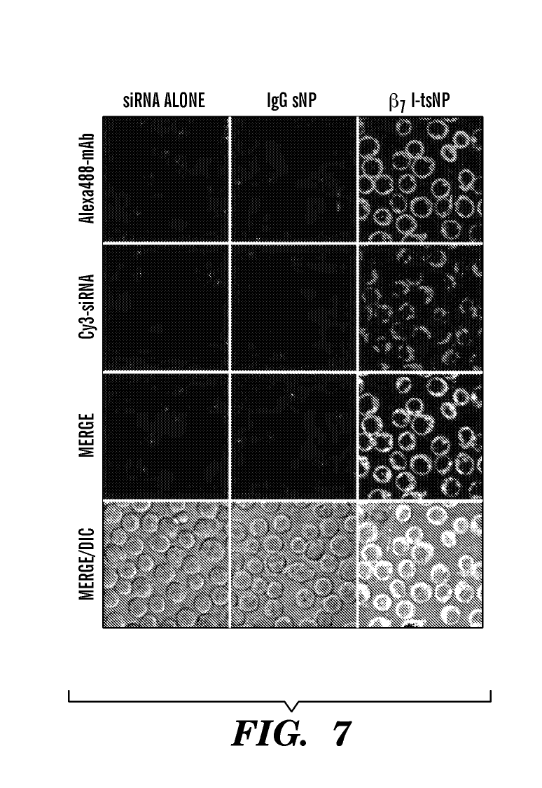

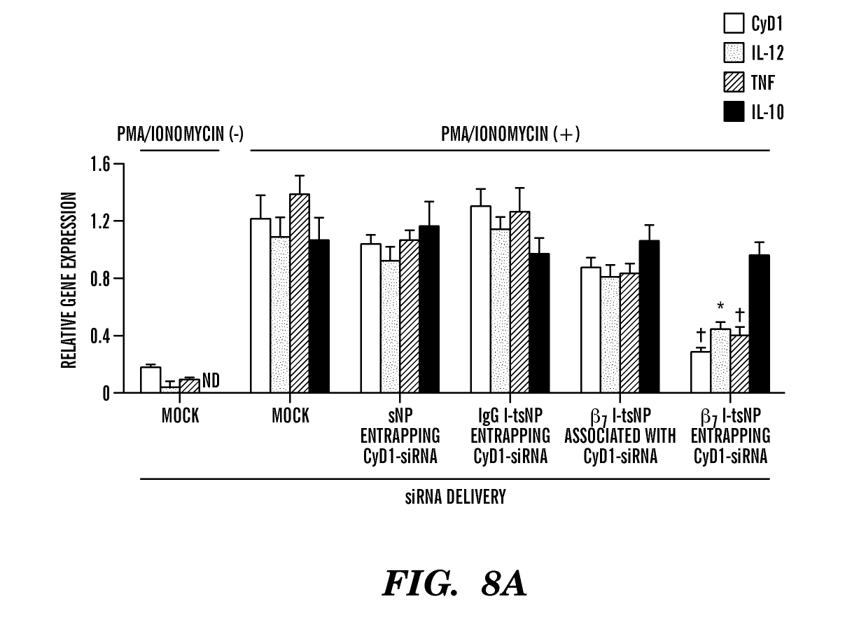

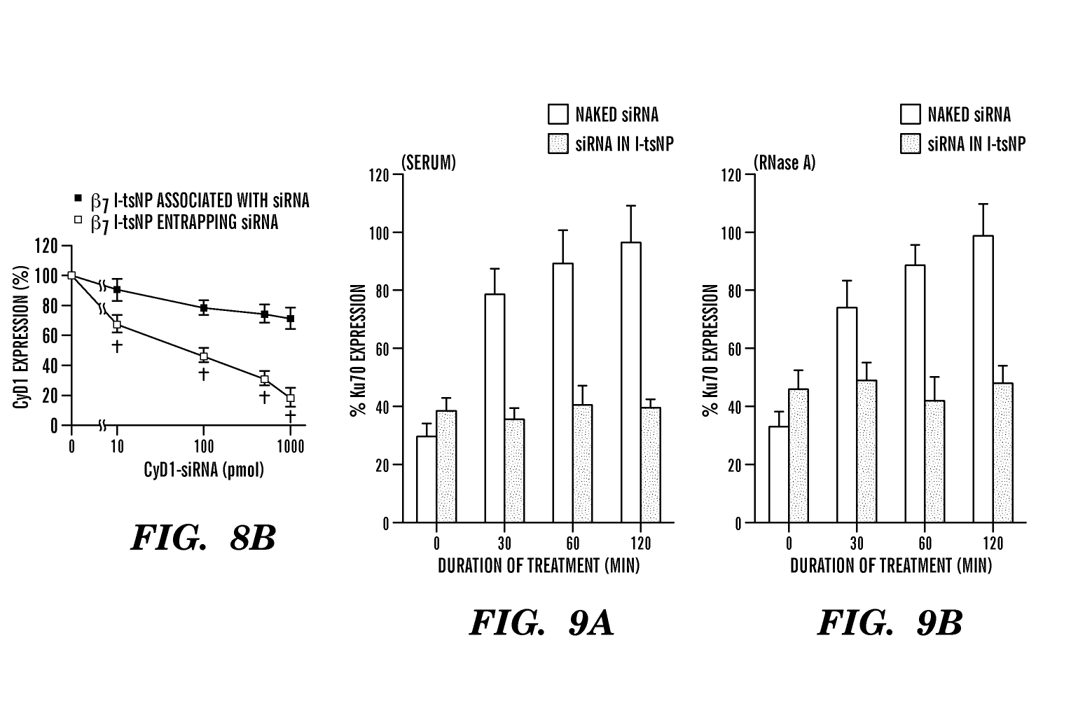

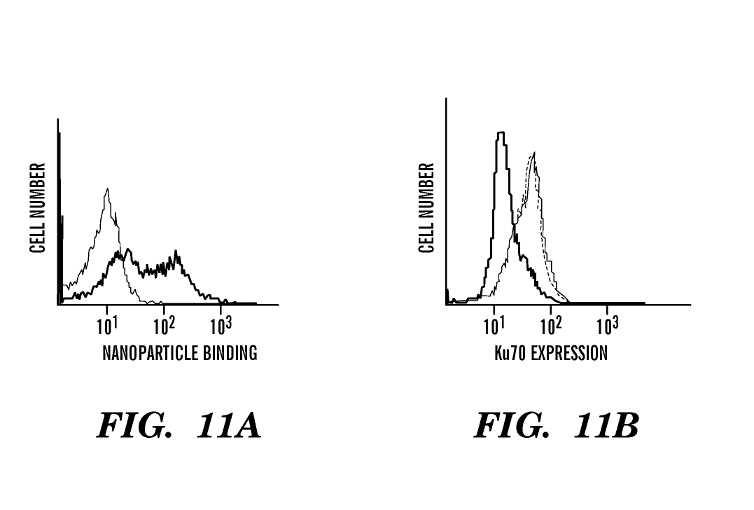

Cy3-siRNA encapsulated within β7 I-tsNP was efficiently bound and delivered to wild-type (WT) but not to β7 integrin knockout (KO) splenocytes (FIG. 2A). Upon cell binding, β7 I-tsNP readily internalized and released Cy3-siRNA to the cytoplasm of both WT splenocytes (FIG. 2B) and the TK-1 lymphocyte cell line (**FIG. 7). Neither naked siRNA nor isotype control IgG-attached stabilized nanoparticles (IgG-sNP) delivered Cy3-siRNA above background levels (FIG. 2, A and B). Using siRNA to Ku70, a ubiquitously expressed nuclear protein and reference target, we demonstrated that Ku70-siRNA delivered by β7 I-tsNP induced potent gene silencing in splenocytes, whereas naked or IgG-sNP-formulated Ku70-siRNA did not (FIG. 2C) (additional results in **FIGS. 8 to 11).

To investigate the ability of β7 I-tsNP to silence genes in vivo, 2.5 mg/kg Ku70-siRNAs entrapped in β7 I-tsNP were administered by intravenous injection into mice and Ku70 expression tested in mononuclear leukocytes isolated from the gut and spleen after 72 h (FIG. 2D). Ku70-siRNAs delivered by β7 I-tsNP potently suppressed Ku70 expression in cells from the gut (including lamina propria and intraepithelial lymphocyte compartments) and spleen. No silencing was observed in cells from identically treated β7 integrin KO mice, confirming the specificity to the β7 integrin-expressing cells. Furthermore naked siRNA and that delivered with IgG-sNP failed to induce detectable silencing in WT or KO mice.

The bio-distribution of 3H-hexadecylcholesterol-labeled nanoparticles intravenously injected into healthy or diseased mice suffering dextran sodium sulphate (DSS)-induced colitis was next investigated (FIG. 2E). IgG-sNP showed very little distribution to the gut regardless of the presence of colitis. By contrast, a substantial portion (˜10%) of β7 I-tsNP spread to the gut in healthy mice. Remarkably, the bio-distribution of β7 I-tsNP to the gut selectively increased ˜3.5-fold in the presence of colitis. Preferential re-distribution of β7 I-tsNP to the inflamed gut is advantageous for delivery siRNAs to treat intestinal inflammation.

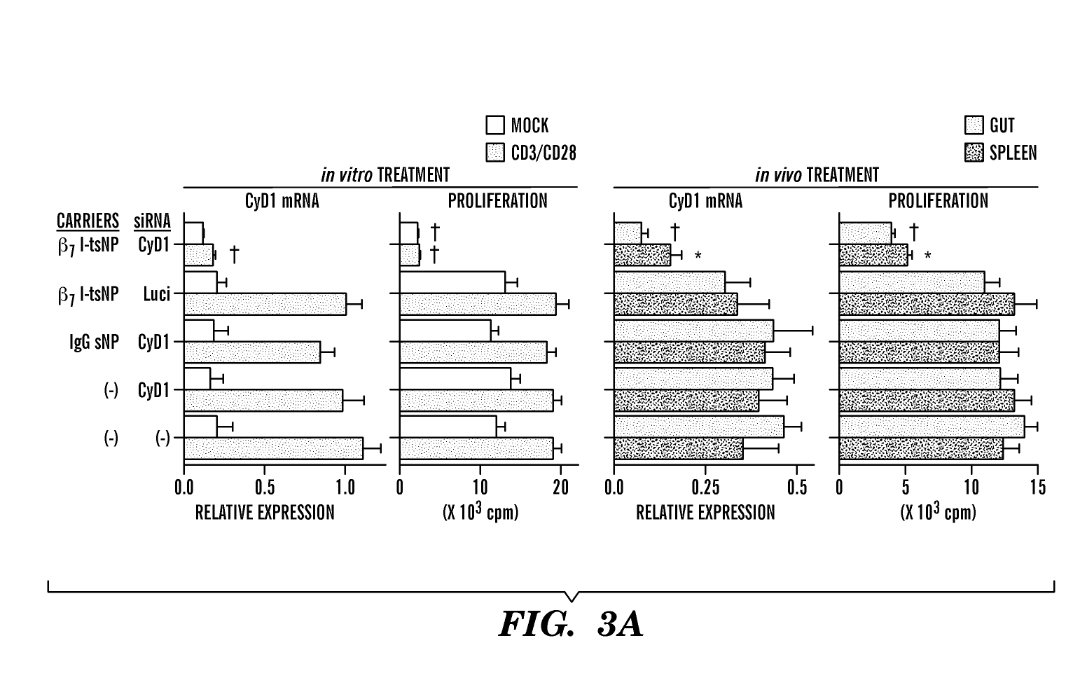

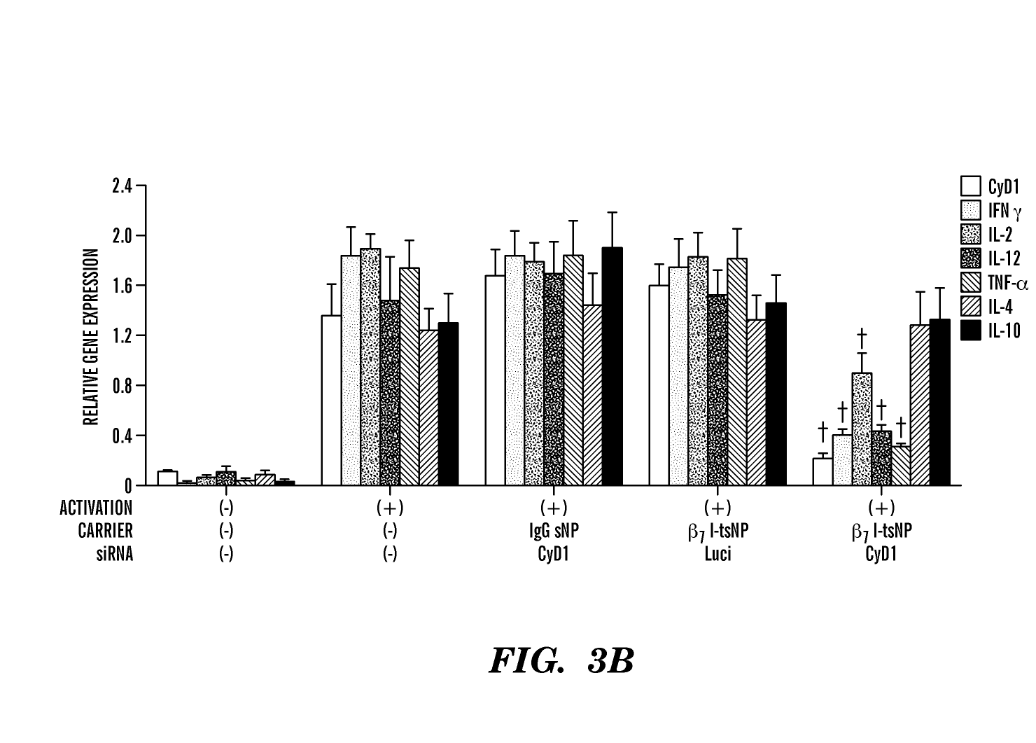

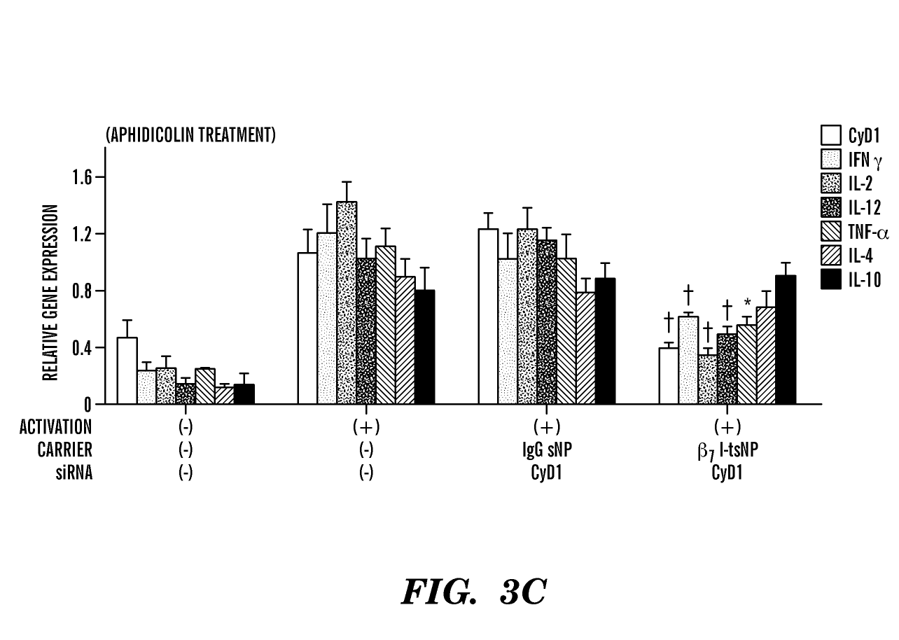

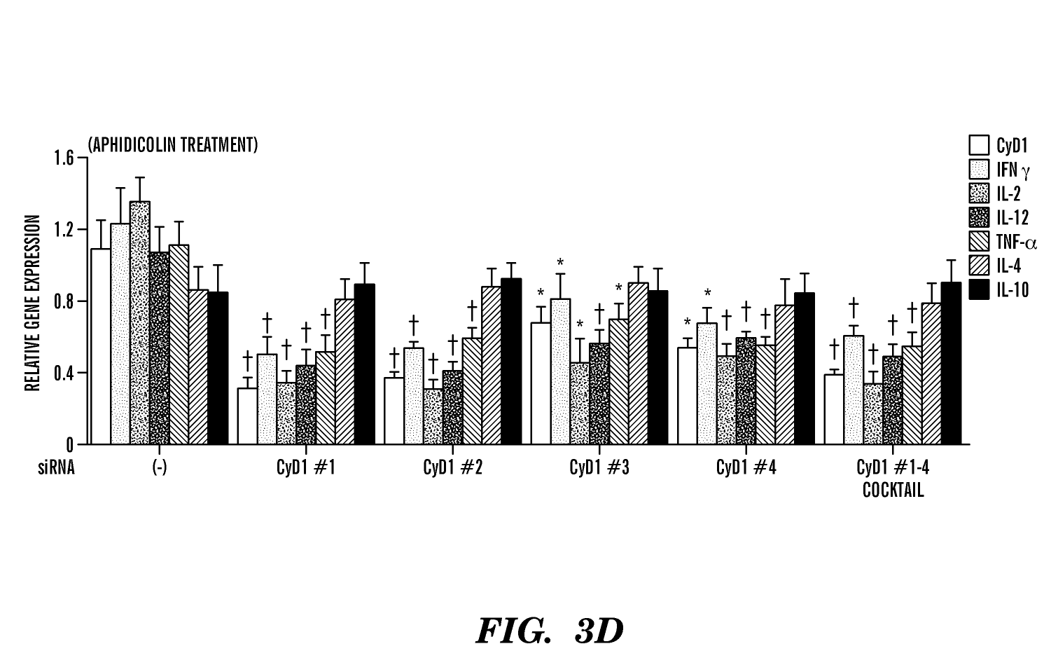

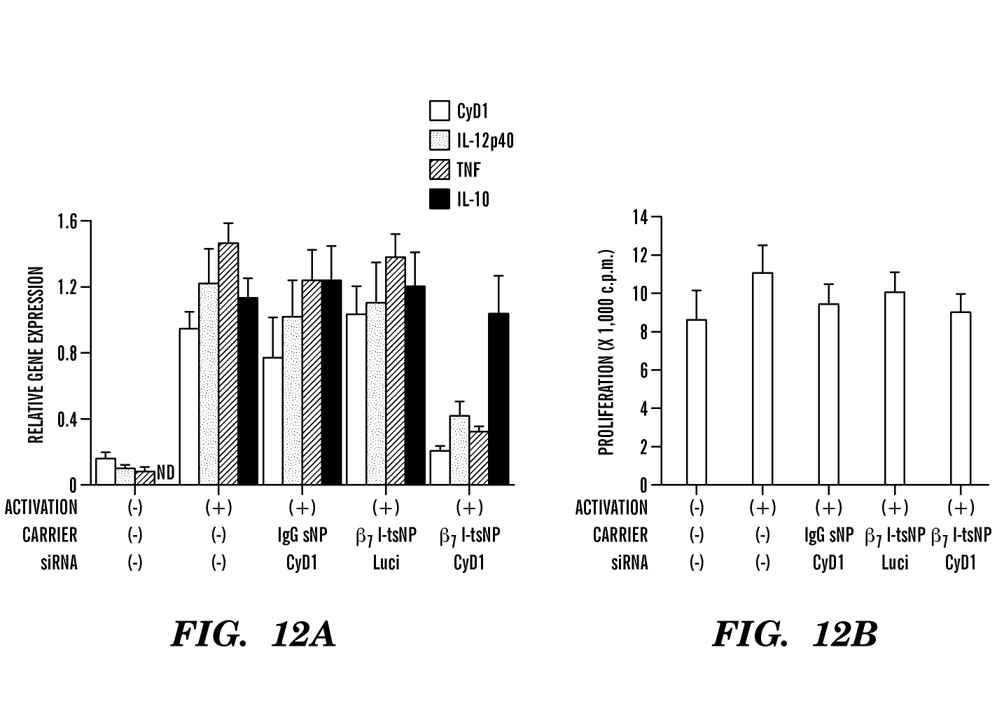

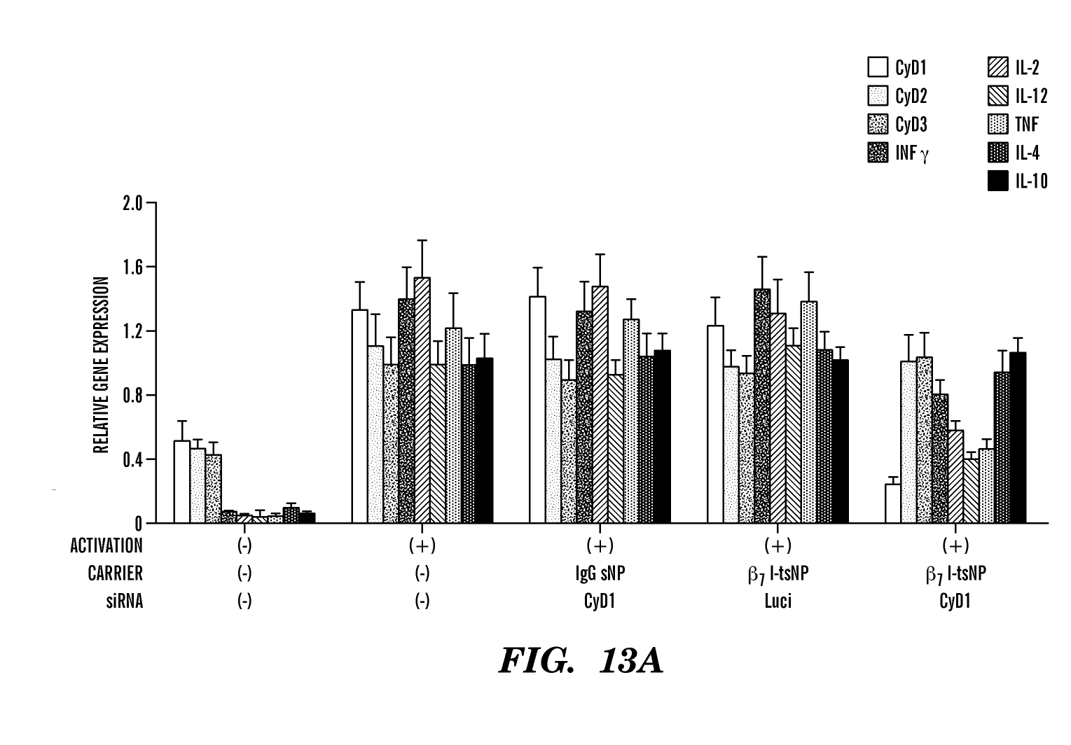

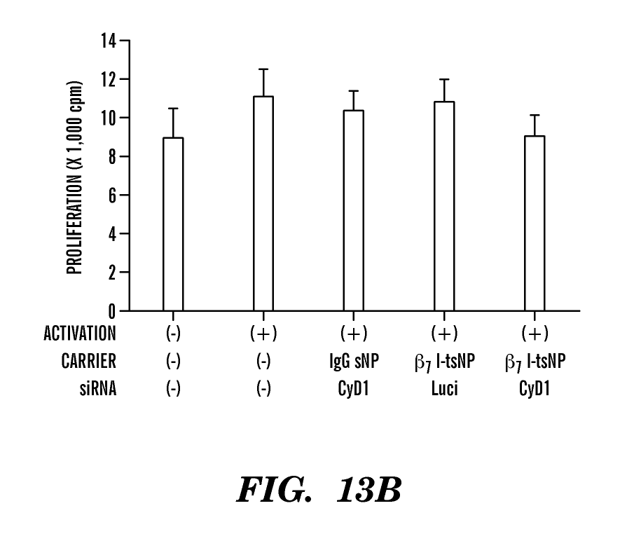

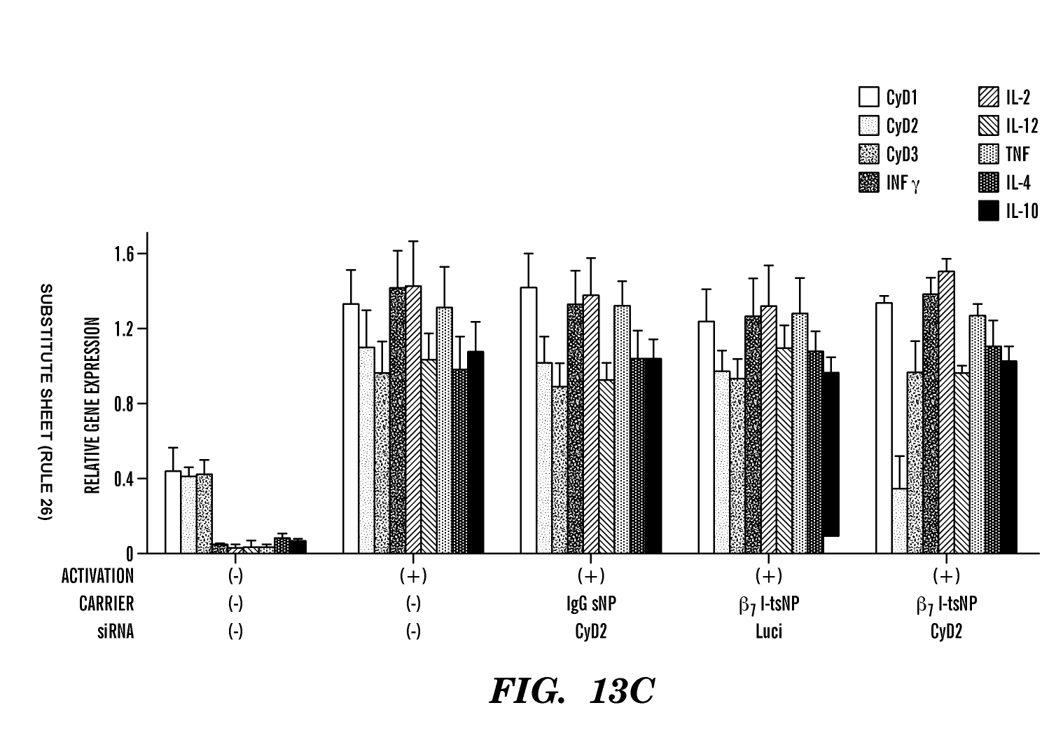

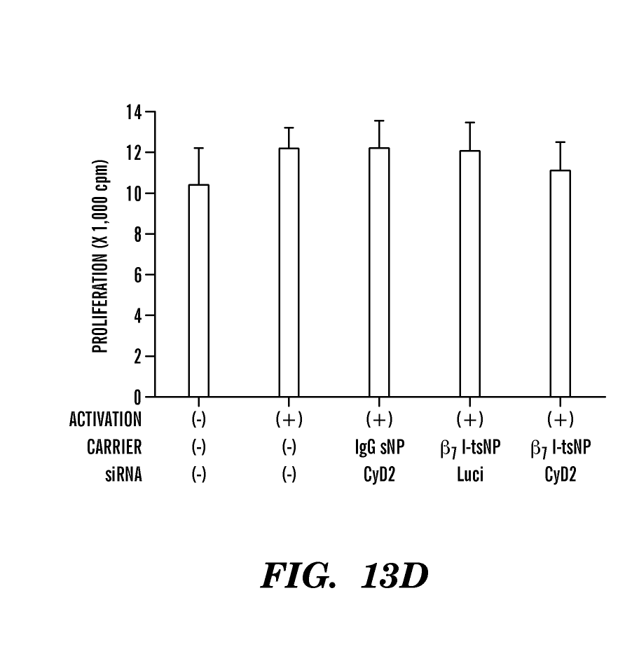

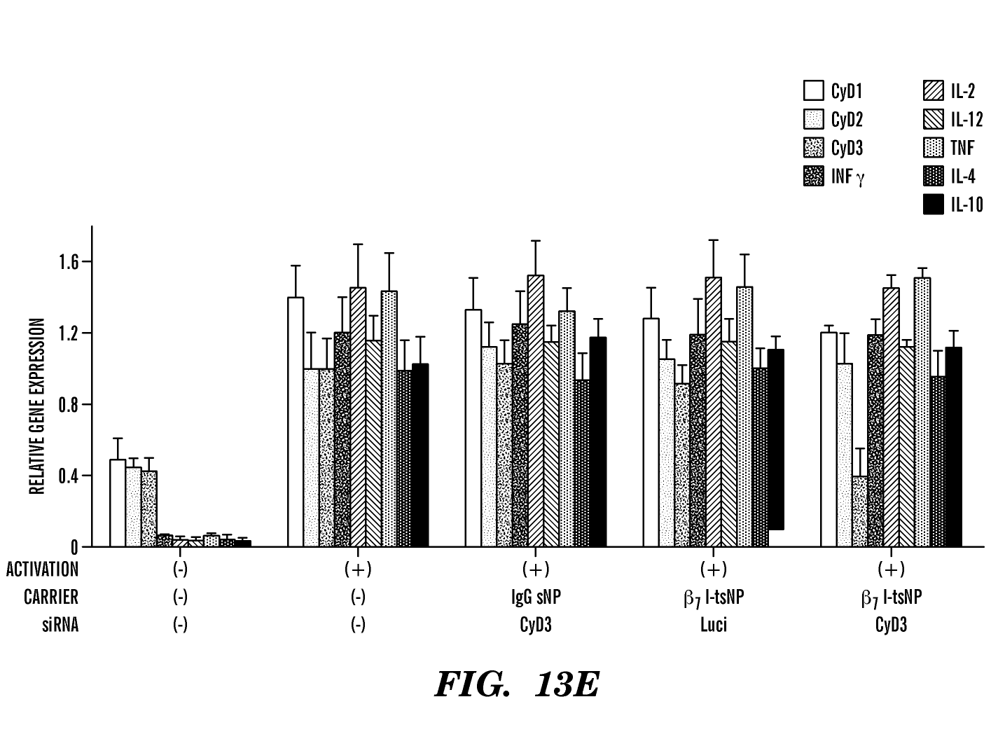

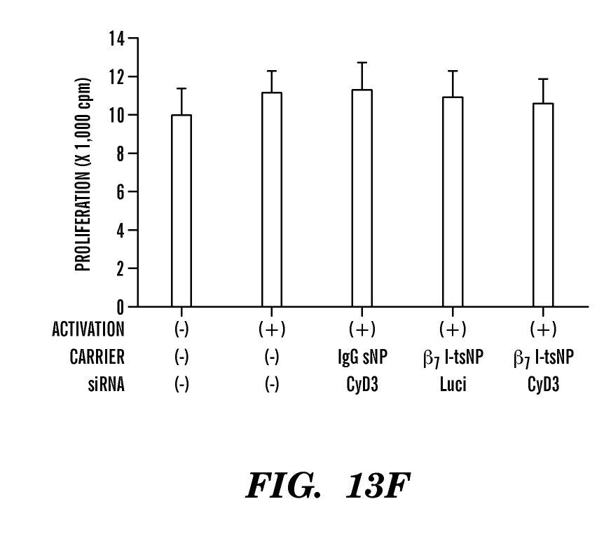

Using β7 I-tsNP, the effects of silencing by CyD1-siRNA were studied. Treatment with β7 I-tsNP entrapping CyD1-siRNA reduced CyD1 mRNA expression in stimulated splenocytes, leading to a potent suppression of proliferation (FIG. 3A). β7 I-tsNP-entrapped CyD1-siRNA (2.5 mg/kg) was then i.v. administered. Three days later, splenic and gut mononuclear leukocytes from mice treated with β7 I-tsNP-entrapped CyD1-siRNA showed a significantly decreased CyD1 mRNA and reduced proliferation (FIG. 3A). Interestingly, while CyD1-knockdown blocked agonist-enhanced expressions of the Th1 cytokines IFN-γ, IL-2, IL-12, and TNF-α, it did not alter those of the Th2 cytokines IL-4 and IL-10, in CD3/CD28- or PMA/iomomycin-stimulated splenocytes (FIGS. 3B and 12) as well as PMA/iomomycin-stimulated TK-1 cells (FIG. 13). The preferential inhibition of Th1 cytokines was not observed with cyclin D2- or cyclin D3 knockdown (FIG. 13). To investigate whether CyD1-knockdown suppressed Th1 cytokine expression independent of its inhibitory effects on cell cycle, TK-1 cells were treated with aphidicolin to arrest cell cycle independent of cyclin D1 status (FIG. 3C). In aphidicolin-treated cells, PMA/iomomycin upregulated mRNA levels of CyD1 as well as Th1 and Th2 cytokines. CyD1-knockdown suppressed selectively Th1 cytokine mRNA expression in aphidicolin-treated and PMA/iomomycin-activated cells (FIG. 3C). This cell cycle-independent suppression of Th1 cytokines was also seen with individual applications of 4 different CyD1-siRNAs that target non-overlapped sequences in CyD1 mRNA (FIG. 3D), excluding that blockade of Th1 cytokines was due to an off-target effect. Thus, CyD1-knockdown preferentially suppresses pro-inflammatory Th1 cytokines independently of changes in cell cycle.

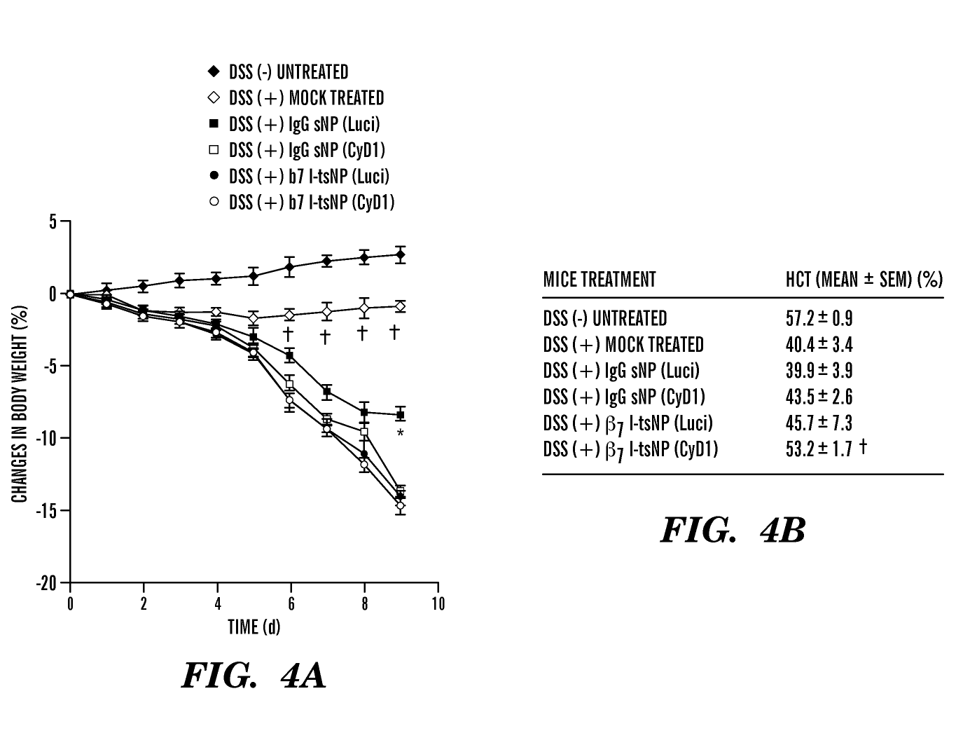

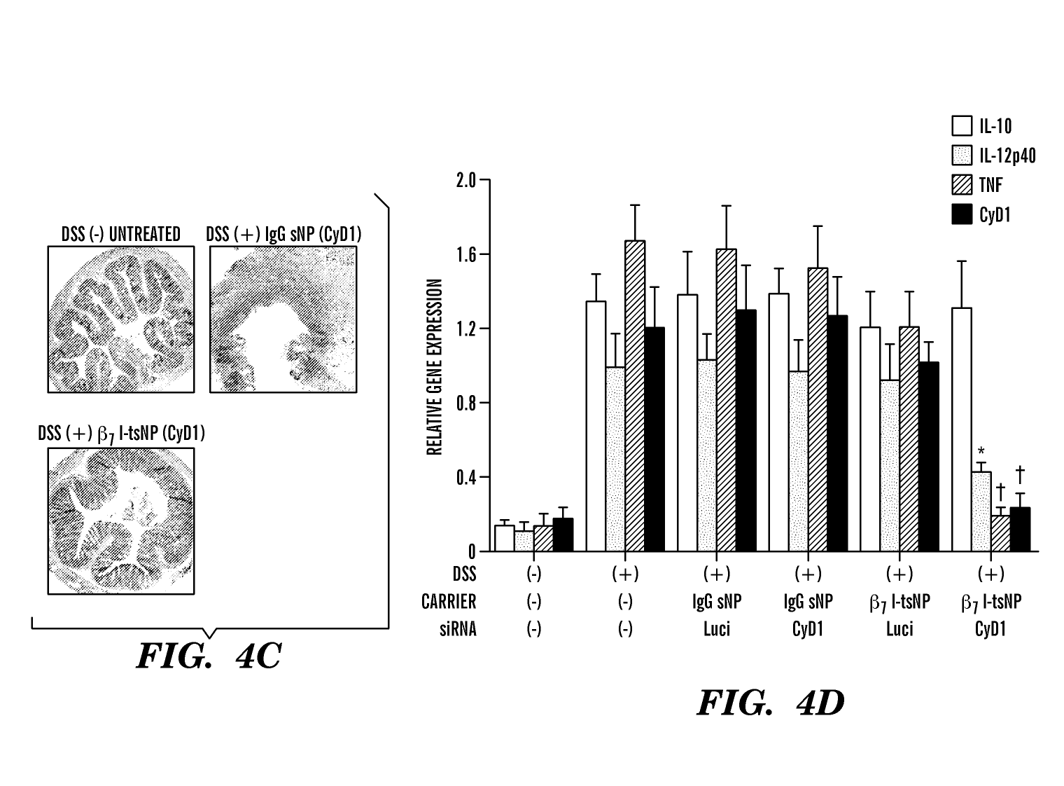

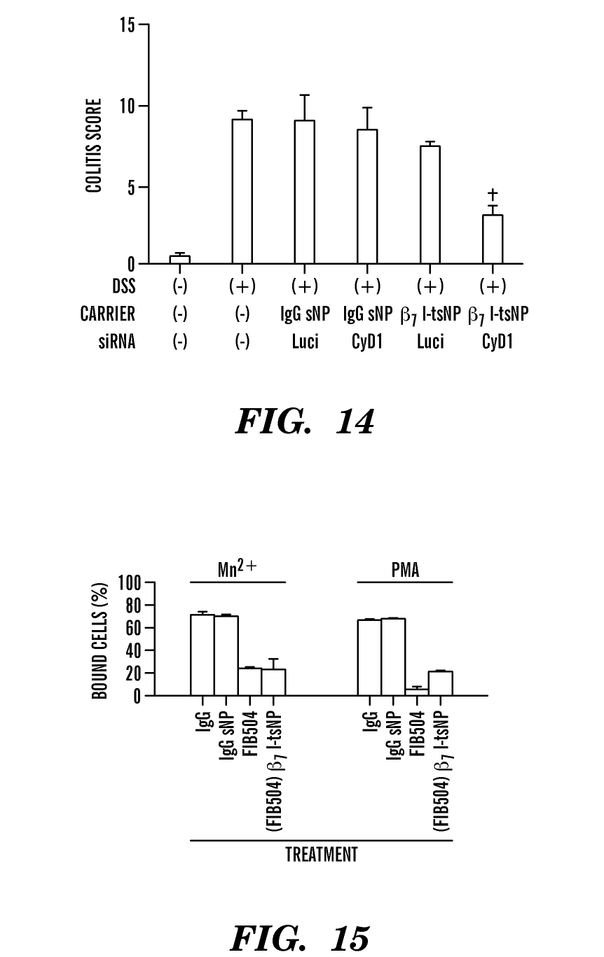

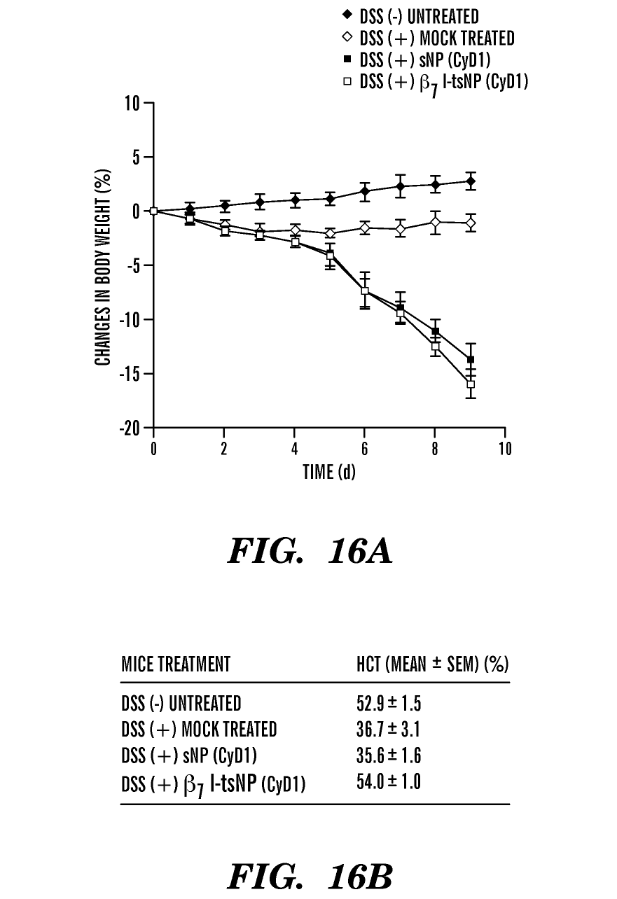

CyD1-knockdown was studied with β7 I-tsNP in vivo using DSS-induced colitis. Mice were intravenously injected 2.5 mg/kg CyD1-siRNA entrapped in β7 I-tsNP or IgG-sNP at days 0, 2, 4, and 6. β7 I-tsNP-delivered CyD1-siRNA potently reduced CyD1 mRNA to a level comparable with that of the uninflamed gut (FIG. 4D). CyD1-knockdown concomitantly suppressed mRNA expression of TNF-α and IL-12, but not IL-10 (FIG. 4D). Remarkably, β7 I-tsNP-delivered CyD1-siRNA led to a drastic reduction in intestinal tissue damage, a potent suppression of leukocyte infiltration into the colon, and a reversal in body weight loss and hematocrit reduction (FIG. 4, A to C, FIG. 14). Importantly, the gut tissue of CyD1-siRNA/β7 I-tsNP-treated animals exhibited normal numbers of mononuclear cells (FIG. 4C), suggesting that CyD1-knockdown does not induce pathologic cell death in the gut. CyD1-siRNAs entrapped in IgG-sNP did not induce silencing in the gut, failing to alter cytokine expression in the gut or reversing manifestations of colitis (FIG. 4, A to C) (additional results in FIGS. 15 and 16).

The anti-inflammatory effects of CyD1-knockdown in colitis are likely to be mediated both by suppressing the aberrant proliferation of mucosal mononuclear leukocytes, and by reducing the expression of TNF-α and IL-12, pro-inflammatory Th1 cytokines that are critical to the pathogenesis of colitis. The Th2 cytokine IL-10 has been shown to suppress inflammation in colitis (13). Thus, the transformation from a relatively Th1-dominant to a more Th2-dominant phenotype appears to represent a critical and unexpected component of the potent colitis inhibition that results from CyD1-knockdown.

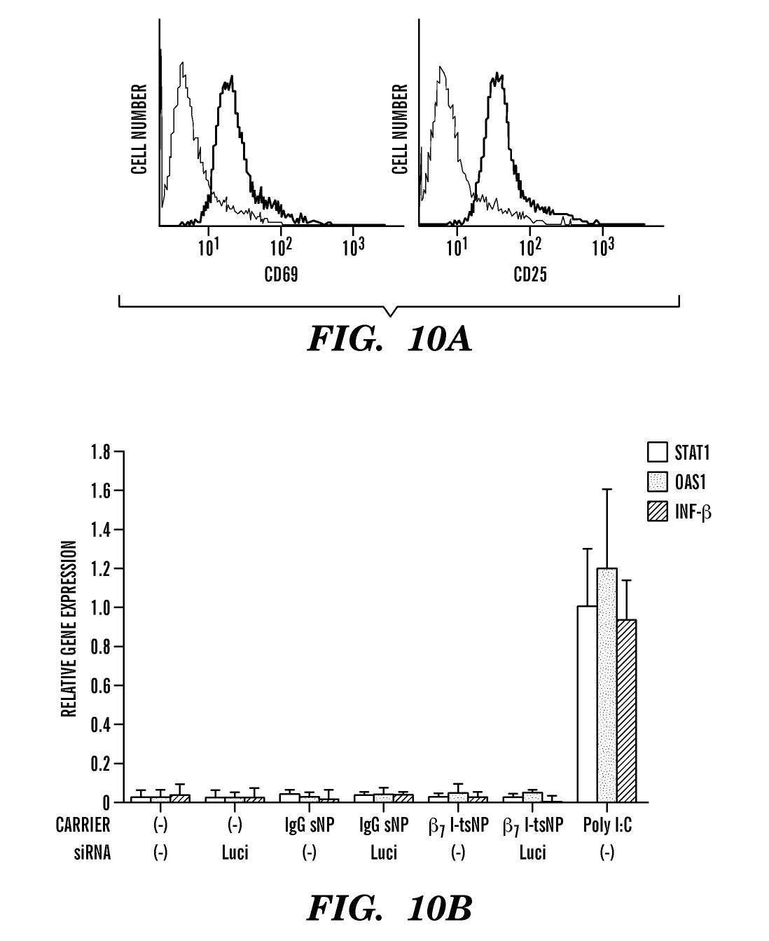

Entrapment of condensed siRNA inside these nanoparticles, in tandem with the targeting of the leukocyte β7 integrin, which readily internalizes bound particles, enabled both highly efficient intracellular delivery and gene silencing in vivo. An effective in vivo siRNA dose of 2.5 mg/kg represents one of the lowest reported to date for systemically targeted siRNA delivery applications (14-19). Compared to other strategies, tsNP offer the combined benefits of low off-target/toxicity and high cargo capacity ˜4000 siRNA molecules per NP). Encapsulation of siRNA within the tsNPs seems to both protect siRNA from degradation (FIG. 9) and prevent triggering on unwanted interferon responses (FIG. 10). Antibodies coated on the outer surface of the NPs provided selective cellular targeting and cell surface integrins proved to be effective antibody targets for both delivery and uptake of tsNP. Thus, the I-tsNP approach can have broad applications for both in vivo drug target validation, and therapeutics.

### REFERENCES

1. E. Iorns, C. J. Lord, N. Turner, A. Ashworth, Nat Rev Drug Discov 6, 556 (2007).
2. D. W. Stacey, Curr Opin Cell Biol 15, 158 (2003).
3. M. Fu, C. Wang, Z. Li, T. Sakamaki, R. G. Pestell, Endocrinology 145, 5439 (2004).
4. R. Yang, W. Bie, A. Haegebarth, A. L. Tyner, Cell Cycle 5, 180 (2006).
5. H. van Dekken et al., Acta Histochem 109, 266 (2007).
6. M. A. Behlke, Mol Ther 13, 644 (2006).
7. D. M. Dykxhoorn, J. Lieberman, Annu Rev Biomed Eng 8, 377 (2006).
8. D. Peer, P. Zhu, C. V. Carman, J. Lieberman, M. Shimaoka, Proc Natl Acad Sci USA 104, 4095 (2007).
9. Z. Ma et al., Biochem Biophys Res Commun 330, 755 (2005).
10. D. Peer, A. Florentin, R. Margalit, Biochim Biophys Acta 1612, 76 (2003).
11. D. P. Andrew et al., J Immunol. 153, 3847 (1994).
12. S. K. Shaw, M. B. Brenner, Semin Immunol 7, 335 (1995).
13. J. O. Lindsay, A. Sandison, P. Cohen, F. M. Brennan, H. J. Hodgson, Dig Dis Sci 49, 1327 (2004).
14. J. Soutschek et al., Nature 432, 173 (2004).
15. D. V. Morrissey et al., Nat Biotechnol 23, 1002 (2005).
16. R. M. Schiffelers et al., Nucleic Acids Res 32, e149 (2004).
17. E. Song et al., Nat Biotechnol 23, 709 (2005).
18. F. Takeshita et al., Proc Natl Acad Sci USA 102, 12177 (2005).
19. J. D. Heidel et al., Proc Natl Acad Sci USA 104, 5715 (2007).
1 D. Peer, A. Florentin, R. Margalit, Biochim Biophys Acta 1612, 76 (2003).
2 D. Peer, R. Margalit, Arch Biochem Biophys 383, 185 (2000).
3 P. Sapra, T. M. Allen, Clin Cancer Res 10, 2530 (2004).
4 D. Peer, Y. Dekel, D. Melikhov, R. Margalit, Cancer Res 64, 7562 (2004).
5 H. G. Enoch, P. Strittmatter, Proc Natl Acad Sci USA 76, 145 (1979).
6 D. Peer, P. Zhu, C. V. Carman, J. Lieberman, M. Shimaoka, Proc Natl Acad Sci USA 104, 4095 (2007).
7 A. D. Judge, G. Bola, A. C. Lee, I. MacLachlan, Mol Ther 13, 494 (2006).
8 M. Kim, C. V. Carman, T. A. Springer, Science 301, 1720 (2003).
9 E. Song et al., Nat Biotechnol 23, 709 (2005).
10 E. J. Park et al., J. Immunol. 171, 4131 (2003).
11 M. F. Neurath et al., J Exp Med 195, 1129 (2002).
12 S. L. Zeger, K. Y. Liang, Biometrics 42, 121 (1986).
13 D. Peer, R. Margalit, Neoplasia 6, 343 (2004).
14 M. A. Behlke, Mol Ther 13, 644 (2006).
15 D. M. Dykxhoorn, J. Lieberman, Annu Rev Biomed Eng 8, 377 (2006).
16 D. P. Andrew et al., J Immunol. 153, 3847 (1994).

TABLE 1
Table 1. Summary of nanoparticle surface
modification and siRNA entrapment.

| | | | siRNA |
|---|---|---|---|
| | mAb¹ | siRNA² | encapsulation |
| Nanoparticle | (molecules/particle) | (molecules/particle) | efficacy³ |
| IgG sNP | 45 ± 15 | 3750 ± 1300 | 78 ± 10 |
| β₇ I-tsNP | 43 ± 17 | 4000 ± 1200 | 80 ± 12 |

The number of mAbs attached to particles was determined as described in Methods using ¹²⁵I-labeled mAbs.
²The number of siRNA entrapped in particles and
³the entrapment efficacy were determined as described in Methods using RiboGreen ™ assay. Data are expressed as the mean ± SEM of at least three independent experiments.

TABLE 2
Diameter and Zeta potential measurements of nanoparticles
before and after an entrapment or a surface-association
of siRNA/protamine complexes.

| Samples | Diameter (Zeta potential) |
|---|---|
| β₇ I-tsNP | |
| −lyophilization/rehydration with water | 107 ± 14 nm (−25.1 ± 4.1 mV) |
| +lyophilization/rehydration with water² | 119 ± 13 nm (−23.7 ± 2.6 mV) |
| siRNA condensed with protamine¹ | 114 ± 7 nm (+13.5 ± 1.2 mV) |
| β₇ I-tsNP entrapping siRNA/protamine² | |
| Time after siRNA addition (min) | |
| 20 | 144 ± 25 nm (−19.8 ± 2.9 mV) |
| 80 | 153 ± 41 nm (−17.9 ± 3.1 mV) |
| 140 | 161 ± 39 nm (−18.1 ± 2.6 mV) |
| β₇ I-tsNP surface-associated | |
| with siRNA/protamine³ | |
| Time after siRNA addition (min) | |
| 20 | 293 ± 35 nm (−8.2 ± 2.4 mV) |
| 80 | 637 ± 98 nm (−5.1 ± 1.7 mV) |
| 140 | 877 ± 101 nm (−4.1 ± 2.1 mV) |

siRNA entrapment, achieved by adding a protamine-condensed siRNA solution to lyophilized β₇ I-tsNP, maintained the nano-dimensions (~150 nm) of particles, and exhibited only a mild neutralization of the β₇ I-tsNP surface charge. By contrast, addition of a protamine-condensed siRNA solution to water-rehydrated β₇ I-tsNP appeared to form growing aggregates, and exhibited a considerable neutralization of the β₇ I-tsNP surface charge. All measurements were performed using a Zetasizer nano ZS instrument (Malvem) at pH 6.7, 10 mM NaCl at 20° C.
¹siRNAs (1 nmol) were mixed with protamine (5 nmol) in H₂O at room temperature for 20 min in RNase-free tubes.
²lyophilized β₇ I-tsNP (1 mg lipid) were rehydrated with an addition of 200 μl water or a protamine-condensed siRNA solution.
³water-rehydrated β₇ I-tsNP (1 mg lipid) were mixed with a protamine-condensed siRNA solution. Data are mean ± SD of four independent measurements.

TABLE 3
Table 3. Hyaluronan maintains the nano-dimensions of particles
during a cycle of lyophilization and rehydration.

| | | | Diameter (nm)¹ | |
|---|---|---|---|---|
| | | | lyophilizatlon/rehydration | |
| Particles | Hyaluronan | mAb | (−) | (+) |
| sNP² | + | — | 94 ± 10 | 105 ± 15 |
| IgG sNP³ | + | IgG | 103 ± 12 | 114 ± 25 |
| β₇ I-tsNP³ | + | FIB504 | 107 ± 14 | 123 ± 24 |
| IgG-NP⁴ | − | IgG | 109 ± 13 | 1,190 ± 670 |
| β₇ I-tNP⁴ | − | FIB504 | 110 ± 21 | 1,330 ± 750 |

¹Particle size was determined using a Malvern Zetasizer nano ZS ™ Zeta potential and Dynamic Light Scattering Instrument (Malvern Instruments Ltd., Southborough, MA). Data are expressed as the mean ± SEM of 6 independent measurements.
²Hyaluronan was covalently attached to DPPE in nanoliposomes.
³Antibodies were covalently attached to hyaluronan, which was covalently attached to DPPE in nanoliposomes.
⁴Antibodies were covalently attached to DPPE in nanoliposomes.

1. (canceled)

2. (canceled)

3. (canceled)

4. (canceled)

5. (canceled)

6. (canceled)

7. A method for treating or preventing inflammation in a subject in need thereof, the method comprising administering an agent that inhibits cyclin D1 to the subject, wherein inflammation is reduced or prevented, to thereby treat or prevent inflammation in the subject.

8. A method of selectively inhibiting Th1-mediated inflammation in a subject in need thereof, the method comprising administering an agent that inhibits cyclin D1 to the subject, to thereby inhibit Th1-mediated inflammation.

9. The method of claim 7 further comprising determining a level of at least one Th1 cytokine in a sample from said individual relative to a standard, wherein said agent is administered if an increased level of at least one said Th1 cytokine is determined.

10. The method of claim 9 wherein the agent reduces the expression of the Th1 cytokine.

11. The method of claim 9 wherein said Th1 cytokine is selected from the group consisting of TNF-α, IL-2, IL-12, IFN-γ and IL-23.

12. The method of claim 7, wherein said agent comprises an antibody, a nucleic acid or a small molecule.

13. (canceled)

14. The method of claim 12, wherein said nucleic acid comprises an interfering RNA.

15. The method of claim 7, wherein said agent comprises a targeting moiety.

16. The method of claim 15 wherein said targeting moiety targets said agent to a leukocyte.

17. The method of claim 16 wherein said targeting moiety binds a cell surface molecule expressed on a target cell.

18. The method of claim 17 wherein said targeting moiety binds an integrin molecule.

19. The method of claim 15, wherein said targeting moiety binds integrin B7.

20. The method of claim 14 wherein said interfering RNA targets a cyclin D1 mRNA for degradation.

21. A method of treating an autoimmune disease or a disorder characterized by or involving a Th1 inflammatory response in a subject in need thereof, the method comprising administering to said subject an agent that inhibits cyclin D1, wherein said Th1 inflammatory response is reduced, to thereby treat the autoimmune disease.

22. The method of claim 21 further comprising determining a level of at least one Th1 cytokine in a sample from said subject relative to a standard, wherein said agent is administered, if an increased level of at least one said Th1 cytokine is determined.

23. The method of claim 22 wherein the at least one Th1 cytokine is selected from the group consisting of TNF-α, IL-2, IL-12, IFN-γ and IL-23.

24. The method of claim 21 wherein said autoimmune disease or disorder is selected from the group consisting of an inflammatory bowel disease, ulcerative colitis, Crohn's disease, celiac disease, autoimmune hepatitis, chronic rheumatoid arthritis, psoriatic arthritis, insulin-dependent diabetes mellitus, multiple sclerosis, Alzheimer's disease, enterogenic spondyloarthropathies, autoimmune myocarditis, psoriasis, scleroderma, myasthenia gravis, multiple myositis/dermatomyositis, Hashimoto's disease, autoimmune hypocytosis, pure red cell aplasia, aplastic anemia, Sjogren's syndrome, vasculitis syndrome, systemic lupus erythematosus, glomerulonephritis, pulmonary inflammation, septic shock and transplant rejection.

25. The method of claim 21 wherein the agent reduces expression of the Th1 cytokine.

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. The method of claim 8 further comprising determining a level of at least one Th1 cytokine in a sample from said individual relative to a standard, wherein said agent is administered if an increased level of at least one said Th1 cytokine is determined.

38. The method of claim 8, wherein the subject has ulcerative colitis.
